(19) United States
(12) Patent Application Publication
(10) Pub. No.: US 2026/0178998 A1
(43) Pub. Date: Jun. 25, 2026

(54) METHOD FOR CONTROLLING THE HANDLING OF BIOLOGICAL MATERIAL

(71) Applicant: Vitrafy Life Sciences Limited, Tasmania (AU)

(72) Inventors: Brent Michael OWENS, Tasmania (AU); Scott Andrew MASON, Tasmania (AU); Sean Patrick CAMERON, Tasmania (AU); Simon Anthony MCALOON, Tasmania (AU)

(21) Appl. No.: 19/127,480

(22) PCT Filed: Nov. 6, 2023
(86) PCT No.: PCT/AU2023/051117

§ 371 Date: May 6, 2025

(30) Foreign Application Priority Data
Nov. 8, 2022 (US) 2022903337

(51) Int. Cl. G06Q 10/06; G16H 10/60; G16H 40/63

(52) U.S. Cl. CPC G06Q 10/06; G16H 10/60; G16H 40/63

(57) ABSTRACT

A method for controlling the handling of a biological material, the method including the steps of: receiving, from a first apparatus of the plurality of apparatuses, a device associated with a user interface for inputting data relating to biological material, or a biological material packaging device, status information for the material and identification information identifying a smart contract associated with the material; querying a distributed ledger to retrieve a current status of a smart contract associated with the material; querying a data storage location that is not a secure distributed ledger to retrieve executable instructions; executing the instructions to process the status information, wherein the processing includes: validating the status information, and determining parts of the status information to store on the distributed ledger; and write to the distributed ledger to: store the determined part or parts of the status information, and update the status of the smart contract.

### TECHNICAL FIELD

This disclosure relates to a method for controlling the handling of a biological material. More particularly, the present disclosure relates to controlling the handling of a biological material by one or more biological material handling apparatuses.

### BACKGROUND

Biological material is collected for a range of purposes including, but not limited to, medical treatments, procedures, diagnoses and research. Depending on the purpose for which the material is collected, it may undergo a number of handling steps, which may include, amongst other things, transporting, processing and preserving the biological material. For example, where the biological material includes cells (e.g., a blood sample), processing of the biological material may include isolation, differentiation or expansion of the cells. In view of this, it is important that at every handling step the biological material is identified with its source, such as a human patient or animal subject.

For example, in the field of personalised and precision medicine such as cell and gene therapies, biological material may be collected from a patient or donor, transported to a processing facility at which it is processed to generate a therapeutic product, before being transported and administered to a patient. Where handling or processing cannot take place at a single facility, it may be necessary to transport the biological material between multiple facilities. In some applications, the nature of the sample and/or the geographical distances between collection points, processing facilities and administration points may necessitate chilling or cryopreservation of the sample prior to storage and transport to avoid the biological material being compromised. Furthermore, due to the personalised nature of such treatments, high confidence in the chain of custody, identity and processing associated with a biological material can be important. While these objectives may be easily achievable on a clinical scale, challenges may arise at a commercial scale where errors in identification and processing are far more likely to occur, and possibly go undetected.

It is desired to address or ameliorate one or more disadvantages or limitations associated with the prior art, or to at least provide a useful alternative.

### SUMMARY

In accordance with at least one embodiment of the present invention there is provided a method for controlling the handling of a biological material by a plurality of biological material handling apparatuses, the method implemented by a processor executing computer program instructions stored in memory and including the steps of:
receiving, from one of:
   a first apparatus of the plurality of apparatuses,
   a device associated with a user interface for inputting data relating to biological material, and
   a biological material packaging device,
status information for the biological material and identification information identifying a smart contract associated with the biological material;
querying a cryptographically secured distributed ledger using the identification information to retrieve the smart contract associated with the biological material, including a current status of the smart contract;
based on the retrieved smart contract and current status of the smart contract, querying a data storage location to retrieve executable instructions, wherein the data storage location is not a secure distributed ledger;
executing the executable instructions to cause the processor to at least:
   process the status information, wherein the processing includes:
      validating the status information, and
      determining a part or parts of the status information to store on the distributed ledger; and
   write to the distributed ledger to:
      store the determined part or parts of the status information, and
      update the status of the smart contract.

In accordance with at least a further embodiment of the present invention there is further provided a method for controlling the handling of an extracted biological material from its initial extraction to its ultimate use, the handling involving multiple stages, the method including executing the above method at each of a plurality of the multiple stages.

In accordance with at least another embodiment of the present invention there is provided a system for controlling the handling of a biological material comprising a plurality of biological material handling apparatuses, a coordination system, a cryptographically secured distributed ledger, and a data storage location. The coordination system includes a processor for executing computer program instructions and a memory storing computer program instructions for execution by the processor. The cryptographically secured distributed ledger stores a smart contract associated with the biological material. The data storage location is not a secure distributed ledger, and stores retrievable computer-executable instructions. The computer program instructions stored in the memory include instructions which, when executed by the processor, cause the coordination system to:
receive status information for the biological material and identification information identifying the smart contract, from one of:
   a first apparatus of the plurality of apparatuses;
   a device associated with a user interface for inputting data relating to the biological material; and
   a biological material packaging device,
query the distributed ledger using the identification information to retrieve the smart contract and a current status of the smart contract; and query the data storage location based on the smart contract and current status to retrieve computer-executable instructions. The computer executable instructions stored in the data storage location include instructions which, when executed by the processor, cause the coordination system to:
process the status information by validating the status information and determining a part or parts of the status information to store on the distributed ledger; and
write to the distributed ledger to store the determined part or parts of the status information and update the status of the smart contract.

In accordance with at least another embodiment of the present invention there is further provided a coordination system for controlling the handling of a biological material, the coordination system including:
at least one processor; and
a memory storing computer program instructions, which when executed by the at least one processor cause the coordination system to perform the above method.

In accordance with the present invention there is further provided a non-transitory, machine-readable storage medium having computer program instructions, which when executed by at least one processor of a coordination system cause the coordination system to perform the above method.

### BRIEF DESCRIPTION OF THE DRAWINGS

Some embodiments of the present invention are hereinafter described, by way of non-limiting example only, with reference to the accompanying drawings, in which:

FIG. 1 is a schematic diagram depicting an embodiment of a system for controlling the handling of a biological material by a plurality of biological material handling apparatuses;

FIG. 2 is a schematic diagram depicting components of an embodiment of a biological material handling apparatus;

FIG. 3 is a schematic diagram depicting components of an embodiment of a packaging device;

FIG. 4 is a flowchart depicting an embodiment of a method for controlling the handling of a biological material by one or more biological material handling apparatuses.

FIG. 5 is a flowchart depicting steps that may be included in some embodiments of the method involving the receipt of historical status information and historical identification information.

FIG. 6 is a flowchart depicting steps that may be included in some embodiments of the method involving the receipt of historical status information and historical identification information.

FIGS. 7 to 9 are schematic diagrams showing examples of a coordination system receiving and processing status information, and identifying subsequent subprocesses to be performed.

FIGS. 10 to 29 are flow charts depicting milestones and subprocesses of a smart contract for controlling the handling of a biological material for the purposes of CAR-T therapy.

### DETAILED DESCRIPTION

In at least some embodiments, the present invention may provide a system and method for controlling the handling of a biological material by one or more of a plurality of biological material handling apparatuses.

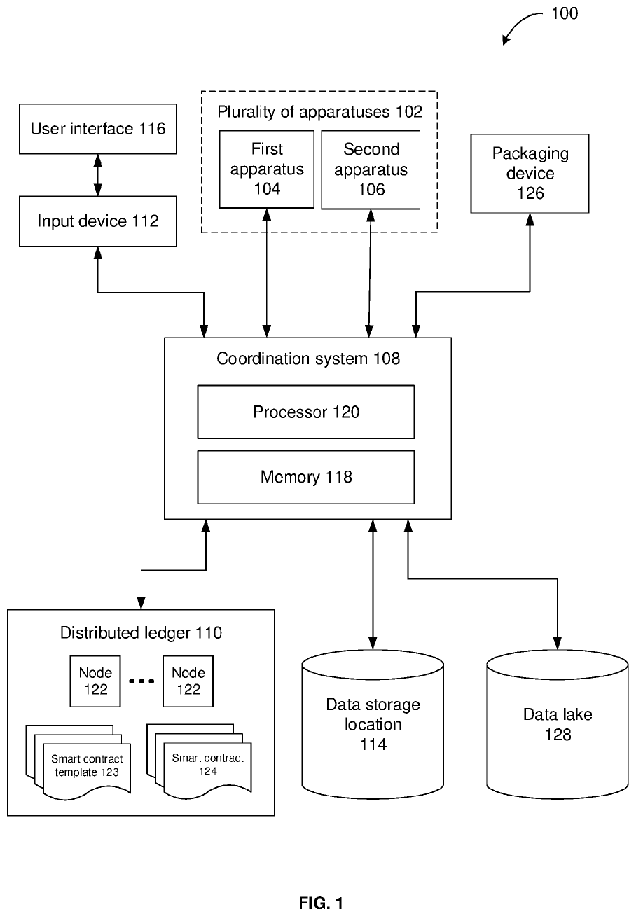

FIG. 1 shows an embodiment of a system 100 for controlling the handling of a biological material by a plurality of biological material handling apparatuses 102. As shown, the apparatuses 102 include a first apparatus 104 and a second apparatus 106. The system 100 includes a coordination system 108, a distributed ledger 110, an input device 112, a data storage location 114 and a data lake 128.

### Coordination System

The coordination system 108 is a computing system including memory 118 and at least one processor 120. The coordination system 108 coordinates the handling of the biological material. To this end, the coordination system 108 is configured to communicate with the distributed ledger 110, the input device 112, the data storage location 114, the data lake 128, each apparatus of the plurality of apparatuses 102, and at least one packaging device 126.

### Input Device

The input device 112 is configured to receive data relating to biological material and communicate that data to the coordination system 108. The input device 112 is associated with a user interface 116 through which the device 112 can receive user input of data relating to biological material, e.g., from a medical professional.

### Distributed Ledger

The distributed ledger 110 includes a plurality of distributed nodes 122. The nodes 122 are organized as a peer-to-peer network in which each node 122 may connect to one or more of the other nodes 122 using a peer-to-peer communication protocol. The configuration of connections between the nodes 122 may change over time, with the nodes that form part of the network at any given point in time communicating using the peer-to-peer protocol. At least one of the nodes 122 connects to the coordination system 108. The distributed ledger 110 is preferably a secure distributed ledger, and more preferably a cryptographically secured distributed ledger. Preferably, the distributed ledger 110 is a decentralised distributed ledger. The distributed ledger 110 may be a public distributed ledger or a private distributed ledger. Use of a private distributed ledger may allow one or more biological material handling apparatuses to host at least one node 122 of the distributed ledger 110, as described herein. In embodiments, the distributed ledger 110 may be a blockchain network (also referred to as a ‘blockchain’), such as a private blockchain network. In embodiments, the distributed ledger 110 may include (at least in part) a public blockchain network.

The coordination system 108 is configured to write to the distributed ledger 110 to store information, which includes storing information representing or derived from the mentioned information being stored to the distributed ledger 110. Where this specification refers to information being written to a distributed ledger, it should be interpreted as including writing that information to the distributed ledger, or writing to the distributed ledger any information representing or derived from that information.

### Smart Contracts

Among other things, the distributed ledger 110 stores one or more smart contract templates 123 associated with handling of biological materials. Each smart contract template 123 defines executable logic which can be executed by the coordination system 108. The executable logic of a given smart contract template 123 defines a set of rules in the form of milestones and corresponding subprocesses that are to take place in relation to biological material. Each smart contract template 123 may correspond to a respective kind or category of biological material and/or a process related to a kind or category of biological material. For example, a particular template 123 may correspond to CAR-T cell therapy (where the biological material includes peripheral blood mononuclear cells) or to assisted reproductive therapy (where the biological material may include sperm, eggs or embryos), or any other process or therapy involving the use of biological material (which could include, but is not limited to, extracted biological material and samples of biological material). Each template 123 stored on the distributed network 110 may be associated with a corresponding unique identifier (“template identifier”).

The distributed ledger 110 also stores one or more smart contracts 124, each of which is an instance of a smart contract template 123. A smart contract 124 is initialised on the distributed ledger 110 using a relevant template 123 in response to user input. The user input indicates the desired smart contract template 123 to be used to initialise the smart contract 124. The coordination system 108 receives the user input (e.g., via the input device 112 as described herein) and accordingly sends a request to the distributed ledger 110 to initialise and store the smart contract 124 using the relevant template 123. The coordination system 108 may assign a unique identifier to the initialised smart contract 124 (“smart contract identifier”), which is stored along with the smart contract 124 on the distributed ledger 110.

By processing a smart contract 124 that is generated based on a template 123 (i.e., a smart contract instance 124 generated from a smart contract template 123), the coordination system 108 is able to maintain a record of data relating to the biological material, and control operations of relevant apparatuses 102 to handle the biological material, e.g., for storage, transport, processing and the like. To control relevant apparatuses 102, the coordination system 108 can determine one or more commands or computer executable instructions, and transmit such commands or executable instructions to each of the plurality of apparatuses 102.

### Biological Material Handling Apparatuses

Each of the plurality of apparatuses 102 is a biological material handling apparatus. Exemplary biological material handling apparatuses include but are not limited to: cryopreservation apparatuses for cryopreserving biological material by cooling to very low temperatures (appropriate temperatures which may include temperatures below the liquid-to-solid phase change temperature), thawing apparatuses for thawing biological material such as cryopreserved biological material, transport apparatuses for transporting the biological material under controlled conditions (e.g., a transport apparatus configured to transport the biological material using a drone or in temperature controlled conditions), storage apparatuses for storing the biological material under controlled conditions, bioreactor apparatuses, and analysis apparatuses (such as cell counter apparatuses for determining a number and/or type of cells present in the biological material and in vitro diagnostic apparatuses).

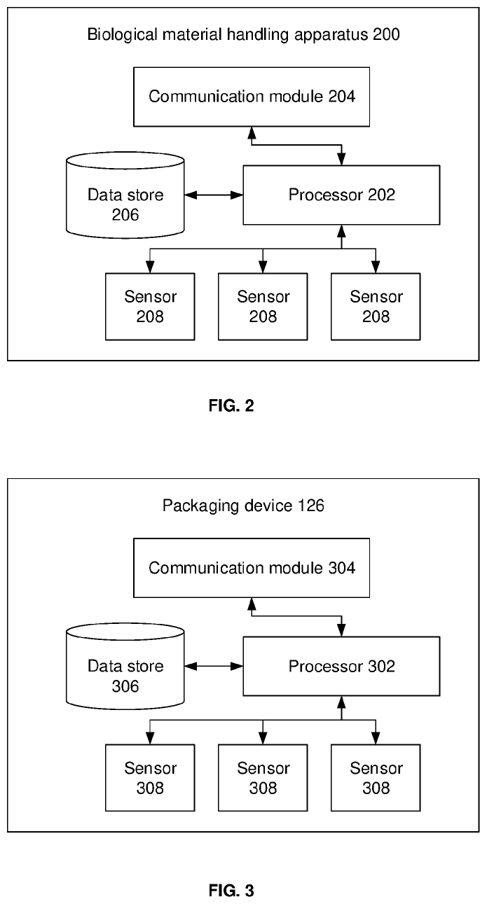

Components an exemplary biological material handling apparatus 200 are schematically represented in FIG. 2. The apparatus 200 includes a processor 202, a communication module 204, a data store 206, and one or more sensors 208 (three sensors are depicted by example only). The processor 202 controls the operation of the apparatus 200 and is configured to execute an application that manages the collection, processing, storage (to the data store 206) and transmission of data received from any of the sensors 208. The processor 202 generates status information relating to operations carried out by the apparatus 200 in relation to the biological material being handled by the apparatus 200, the status information being transmitted to the coordination system 108 by the communication module 204.

The status information can include monitoring information for the biological material. Monitoring information can include, for example, at least one of temperature monitoring information relating to a current temperature of the biological material, location monitoring information relating to a current location of the biological material, volume monitoring information relating to a volume of the biological material, and cell count information relating to a number of cells associated with the biological material.

The communication module 204 performs communications between the apparatus 200 and the coordination system 108 over at least one network, which may be a local area network (LAN) (such as an Ethernet or Wi-Fi network), a wide area network (WAN) (such as a 4G or 5G cellular network) or a personal area network (PAN) (which may include Bluetooth, ZigBee, Thread, Matter, Z-Wave, RFID or Wireless USB, or other short-range or mesh communication systems). In this regard, the coordination system 108 may be configured to communicate with each of the plurality of apparatuses 102 through a mesh network. The coordination system 108 may also be configured to directly communicate with each of the plurality of apparatuses 102. Where the communication at least partly uses a mesh network, at least a portion of the apparatuses 102 can also communicate directly with one another using their respective communication modules 204. Data communicated between the apparatus 200 and the coordination system 108 via the communication module 204 may be encrypted. The communication module 204 may communicate data to the coordination system 108 using an Application Programming Interface (API) and a unique API key generated for the apparatus 200. The communication module 204 may also communicate data to the coordination system 108 by directly writing to an open communications port of the coordination system 108, or by any other appropriate (and preferably secure) means.

Each of the sensors 208 captures sensor data relating to biological material being handled by the apparatus 200. For example, a given sensor 208 may be one of the following (although the given configuration of sensors may depend on the apparatus):
a) a temperature sensor for monitoring a temperature of the biological material being handled by the apparatus 200;
b) a location sensor for monitoring a location of the biological material being handled by the apparatus 200;
c) a volume sensor for monitoring a volume of the biological material being handled by the apparatus 200; and
d) a cell count sensor for monitoring a cell count of the biological material being handled by the apparatus 200.

Any other appropriate sensor may be included in the biological material handling apparatus 200.

The processor 202 collects and processes sensor data received from each of the sensors 208 to generate status information, including monitoring information, which can be transmitted to the coordination system 108 by the communication module 204.

The processor 202 may store generated status information, including monitoring information, in the data store 206 of the apparatus 200. This may be in addition to transmitting the status information to the coordination system 108 using the communication module 204, such that the stored data can be later referred to, e.g., for data integrity or recovery purposes. The status information can also be stored in the data store 206 before it is transmitted to the coordination system 108 using the communication module 204, such as where the coordination module 204 is temporarily unable to communicate with the coordination system 108 over the at least one network, e.g., due to being temporarily disconnected from the network.

Thus, in the data store 206 the apparatus 200 can maintain a local database of information generated by the apparatus 200. The status information stored in the data store 206 may be referred to as historical status information. The historical status information can also be transmitted to coordination system 108 by the communication module 204. The maintenance of this local database may aid data capture reconciliation, data integrity and data recovery.

Each apparatus 200 may be associated with a unique identifier (“apparatus identifier”). As is further described herein, the apparatus identifier can be used when transmitting status information from the apparatus 200 to the coordination system 108 to allow the coordination system 108 to identify the source of the information.

In some embodiments, at least one apparatus of the plurality of apparatuses 102 is configured to host at least one node 122 of the distributed ledger 110. Accordingly, spare processing capacity inside one or more of the apparatuses can be utilised by the distributed ledger 110. The coordination system 108 may be configured to determine whether the apparatus has sufficient processing and storage capacity to host the at least one node 122 of the distributed ledger. The coordination system 108 may also ensure that the apparatus also has sufficient processing and storage capacity to also carry out primary functions, such as controlling the operation of the apparatus to handle biological material, in addition to acting as a node. For example, the coordination system 108 may determine whether the apparatus has sufficient processing and storage capacity to host the at least one node 122 based on status information in the form of operational monitoring information that is receives from the apparatus. The operational monitoring information may include instantaneous and/or historical CPU utilisation, memory usage and storage utilisation of the apparatus. The operational monitoring information may be received periodically from the apparatus, e.g., every 10 seconds.

If the coordination system 108 determines that the apparatus has sufficient processing and storage capacity to host the at least one node 122 without compromising its capacity to handle biological material, then the coordination system 108 can allow the apparatus to host the at least one node 122, such as by transmitting instructions to the apparatus to host the at least one node 122. Once the apparatus is hosting the at least one node 122, if the coordination system 108 determines that the apparatus no longer has sufficient processing and storage capacity to host the at least one node 122 without compromising its capacity to handle biological material (a determination which may be made based on the received operational monitoring information), then the coordination system 108 can disable the apparatus from hosting the at least one node 122, such as by transmitting instructions to the apparatus to cease hosting the at least one node 122.

### Data Storage Location and Data Lake

In the system 100 shown in FIG. 1, the data storage location 114 stores executable instructions that, when executed by the coordination system 108, cause the processor 120 of the coordination system 108 to perform at least the following:
(i) process the status information, the processing including validating the status information and determining a part (or parts) of the status information to store on the distributed ledger 110;
(ii) write to the distributed ledger 110 to store the determined part (or parts) of the status information; and
(iii) write to the distributed ledger 110 to update the status of the smart contract 124.

An authorised administrator is able to edit executable instructions stored in the data storage location 114 and add executable instructions to the data storage location 114 at any time. While the executable instructions are retrieved and executed in accordance with the smart contract 124, they need not be committed to the distributed ledger 110. This may allow the executable instructions to be more easily modified by the authorised administrator, who has access to the data storage location 114 but may not have access to the distributed ledger 110. Furthermore, where the distributed ledger 110 is an immutable distributed ledger such as a blockchain network, the storage of the executable instructions in the data storage location 114 rather than the distributed ledger 110 avoids the executable instructions being immutably committed to the distributed ledger 110, and the associated computational/storage resources that would be required in the event that any executable instructions require modification. In this regard, if the executable instructions were to be stored on the distributed ledger 110, each node 122 would be required to maintain a separate copy of the executable instructions. Additionally, any modification to the executable instructions stored on the distributed ledger 110 would require the distributed ledger 110 to ensure that consensus is reached among the nodes 122, such as by the nodes 122 executing a consensus protocol. Furthermore, if the executable instructions were immutably committed to the distributed ledger 110, then any modification to the executable instructions stored on the distributed ledger would require the both the modified executable instructions and the original executable instructions to be stored on the distributed ledger 110. However, since the executable instructions are stored in the data storage location 114, only a single record of the executable instructions is required and the data need not be maintained in more than one location. It is also unnecessary for the data storage location 114 to execute a consensus protocol or the like when modifications are made to the executable instructions. Meanwhile, it is still possible to maintain security of the executable instructions stored in the data storage location 114 such as by requiring special permissions to modify data stored in the data storage location 114.

The coordination system 108 can query the data storage location 114 to retrieve executable instructions stored therein.

The data lake 128 is a data storage location in which the coordination system 108 stores status information and identification information received from any of the input device 112, plurality of apparatuses 102 and packaging device 126. The coordination system 108 may also store data that it generates when executing the executable instructions retrieved from the data storage location 114 in the data lake 128.

The data storage location 114 and the data lake 128 can each include any suitable data storage mechanisms, such as local data stores, cloud storage, or distributed file systems (DFS) (e.g., InterPlanetary File System (IPFS)). Although FIG. 1 shows the data storage location 114 and data lake 128 separately, the data storage location 114 and data lake 128 can be a single data storage location.

### Packaging Device

The packaging device 126 is for receiving and containing the biological material, such as while it is being processed, stored and/or transported to and between any of the plurality of apparatuses 102. For particularly sensitive biological materials, the packaging device 126 may be configured for storage, preservation and/or thawing of biological material contained within the packaging device 126. For example, the packaging device 126 may be configured to contain the biological material while the biological material is being preserved, cooled or thawed by one of the plurality of apparatuses 102. The packaging device 126 may include an RFID tag that stores identification information of the packaging device 126. The packaging device 126 may include one or more thermal contours, where in use the biological material is distributed between sub-compartments of the packaging device 126 and flow of a heat exchange fluid can be directed by the thermal contours to improve heat transfer between the heat exchange fluid and the biological material contained in the sub-compartments. Examples of suitable packaging devices are described in Australian Provisional Application No. 2021904254, the entire disclosure of which is incorporated herein by reference.

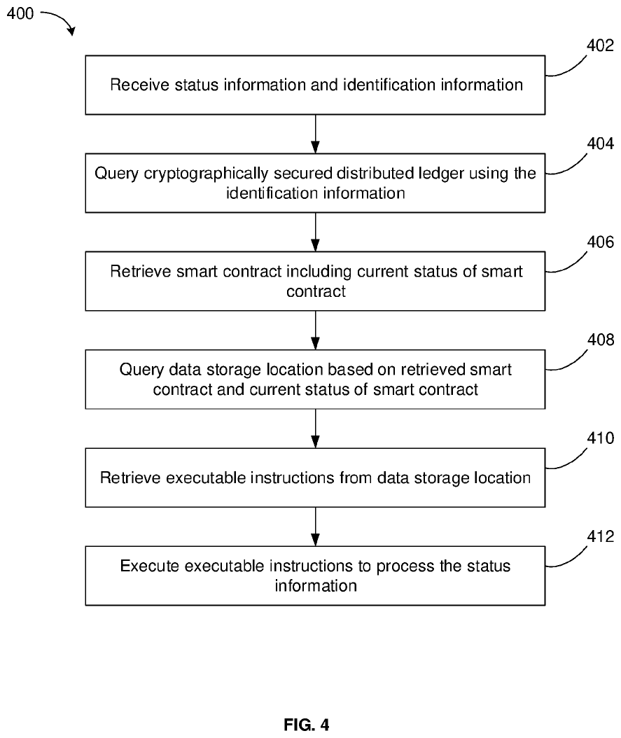

Components of an exemplary packaging device 126 are schematically represented in FIG. 3. The packaging device 300 includes a processor 302, a communication module 304, a data store 306, and one or more sensors 308 (three sensors are depicted by example only). The communication module 304 performs communications between the packaging device 126 and the coordination system 108 over at least one network, e.g., a LAN and/or WAN as described in relation to the communication module 204 of the biological material handling apparatus 200. Data communicated between the packaging device 126 and the coordination system 108 via the communication module 304 may be encrypted. The communication module 304 may communicate data to the coordination system 108 using an Application Programming Interface (API) and a unique API key generated for the packaging device 126.

Each of the sensors 308 captures sensor data relating to biological material being stored by the packaging device 126. For example, a given sensor 308 may be one of the following:
e) a temperature sensor for monitoring a temperature of the biological material being stored by the packaging device 126;
f) a location sensor for monitoring a location of the biological material being stored by the packaging device 126;
g) a volume sensor for monitoring a volume of the biological material being stored by the packaging device 126;
h) a cell count sensor for monitoring a cell count of the biological material being stored by the packaging device 126;
i) a shock sensor for monitoring impacts to the packaging device 126.

The processor 302 collects and processes sensor data received from each of the sensors 308 to generate status information, in the form of monitoring information, which can be transmitted to the coordination system 108 by the communication module 304.

Similarly to the apparatus 200 described above, the processor 302 may store generated monitoring information to the data store 306 of the packaging device 126, such that the stored data can be later referred to, e.g., for data integrity or recovery purposes. Thus, in the data store 306 the packaging device 126 can maintain a local database of information generated by the packaging device 126. The monitoring information stored in the data store 306 may be referred to as historical monitoring information. The historical monitoring information can also be transmitted to coordination system 108 by the communication module 304. The maintenance of this local database may aid data capture reconciliation, data integrity and data recovery.

### Biological Material and Applications

The biological material can include one or more of the following:
a) whole blood;
b) blood components, such as blood platelets, red blood cells, white blood cells, plasma and other blood products;
c) stem cells, such as haematopoietic stems cells, mesenchymal stem cells and embryonic stem cells;
d) modified cells;
e) engineered cells
f) gametes, such as egg cells and sperm;
g) blastocytes
h) oocytes;
i) organs, including parts thereof; and
j) tissue.

It should be appreciated that the examples of biological materials identified herein are not intended to be an exhaustive list, and the packaging may also be used in the preservation of other biological materials.

The system and method described herein may be used to control the handling of a biological material for various purposes. For example, the system and method may be used to control the handling of biological material to be used in therapeutic treatments and cellular therapies.

### Method

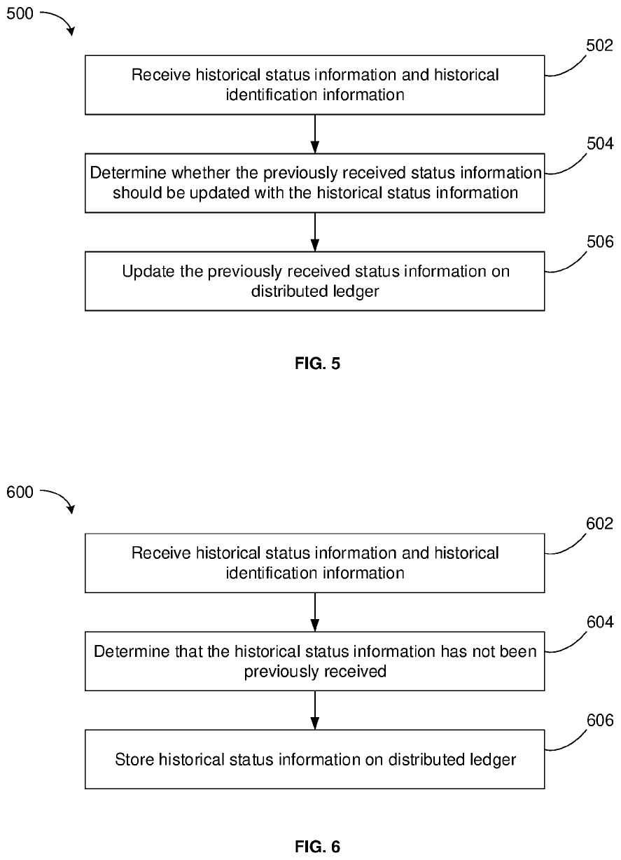

FIG. 4 is a flowchart showing an embodiment of a method 400 for controlling the handling of a biological material by a plurality of biological material handling apparatuses. In this embodiment, the method 400 is implemented by the processor 120 of the coordination system 108. The method 400 begins at step 402.

At step 402, status information for the biological material and identification information identifying the biological material are received at the coordination system 108. The status information and the identification information are received from at least one of: the first apparatus 104 of the plurality of apparatuses 102, the input device 112 associated with the user interface 116, and the biological material packaging device 126.

The identification information uniquely identifies the smart contract stored on the distributed ledger 110 which corresponds to the biological material (i.e., the particular instance of the biological material) to be handled by the plurality of apparatuses 102, as well as the intended use or application of the biological material, e.g., Chimeric Antigen Receptor T-Cell (CAR-T cell) therapy for white blood cells or Assisted Reproductive Therapy (ART) for sperm. The identification information may include or correspond to the smart contract identifier for the relevant smart contract.

The identification information may be generated by the coordination system 108 based on user input received from the user interface 116 via the input device 112 that indicates that a new process and hence a new smart contract is to be initiated.

The status information includes information received by the coordination system 108 that relates to the handling of the biological material but is not identification information. For example, the status information may include information indicating a status of the biological material to be handled by the plurality of apparatuses 102. The status information may include information such as a volume of the biological material (e.g., mL or number of cells) and a quality of the biological material. For example, if the biological material is sperm, the status information may indicate a volume, motility and/or grading of the sperm. The status information may include information indicating a status of one or more of the apparatuses 102 that handle the biological material.

The coordination system 108 is able to identify a particular smart contract 124 of all smart contracts 124 stored on the distributed ledger 110 that is required to coordinate the handling of the relevant biological material by inspecting the received identification and status information.

In some embodiments, the coordination system 108 stores the received status information to the data lake 128. The coordination system 108 may store the received status information to the data lake 128 with its associated identification information, so that the status information for a particular smart contract 124 can be subsequently retrieved from the data lake 128 if required.

At step 404, the coordination system 108 queries the distributed ledger 110 to receive the smart contract 124 determined to be required. The query uses the identification information received at step 402. At step 406, in response to the query, the coordination system 108 retrieves the determined smart contract 124 from the distributed ledger 110. The retrieved smart contract 124 is associated with the biological material by means of the identification information, and includes a current status of the smart contract 124. The current status of the smart contract 124 indicates a “current” milestone and/or subprocess, defined by the smart contract 124, which is currently taking place or is next to take place in relation to the associated biological material.

In some embodiments, the identification information includes a smart contract identifier for the required smart contact, as described above, which the coordination system 108 can use to query the distributed ledger 110 for the required smart contract 124.

Once the coordination system 108 has retrieved the smart contract associated with the smart contract identifier, including the current status of that smart contract 124, at step 408 the coordination system 108 queries the data storage location 114, using the smart contract 124 and the current status of the smart contract 114, in order to retrieve executable instructions that define how the current milestone or process (as determined by the current status of the smart contract 124) is to be performed by the coordination system 108.

At step 410, the coordination system 108 receives the executable instructions from the data storage location 114 in response to the query at step 408.

At step 412, the coordination system 108 executes the retrieved executable instructions to perform the current milestone or subprocess. In this regard, execution of the executable instructions causes the processor 120 of the coordination system 108 to at least process the received status information and write to the distributed ledger 110.

Processing the status information at step 412 may include validation of the status information. Validation includes assessment of the status information to determine whether or not it satisfies one or more requirements for the current milestone or subprocess. This may include, for example, determining whether the status information satisfies a required threshold for that status information or falls within a required range for that status information, where the threshold or range is defined by the executable instructions.

Processing the status information at step 412 may include determining a part or parts of the status information to be stored on the distributed ledger 110. The processing may determine that all or only a portion of the status information is required to be stored on the distributed ledger 110, based on content and/or characteristics of the status information. For example, if the status information includes temperature monitoring information having per second temperature measurements, processing the status information may include determining that only one temperature measurement every 60 seconds is to be stored on the distributed ledger 110, unless a difference between consecutive temperature measurements exceeds a predetermined threshold. This may reduce the quantity of data and associated computational resources required to store to the distributed ledger 110, while ensuring relevant information is still committed to the distributed ledger.

By executing the executable instructions, the coordination system 108 may write to the distributed ledger 110 to store the determined part or parts of the status information to the distributed ledger 110.

In some embodiments, writing the part(s) of the status information to the distributed ledger may include submitting a write request to a gatekeeper component (not shown) associated with the distributed ledger 110. The gatekeeper component may also be described as a ‘gateway’, and is a component of the distributed ledger 110 that manages interactions between the coordination system 108 and the distributed ledger 110, including requests from the coordination system 108 to read data stored on the distributed ledger 110 and to write data to the distributed ledger 110.

The write request may include the part(s) of the status information and the relevant smart contract identifier. In response to receiving the write request, the gatekeeper component may execute a process to transform the part(s) of the status information, e.g., so that they are better suited to be stored on the distributed ledger 110, and store the transformed part(s) of the status information to the distributed ledger 110.

As mentioned above, in some embodiments the coordination system 108 may store the status information to the data lake 128. Therefore, if only a part or parts of the status information are stored to the distributed ledger 110 in accordance with the processing at step 412, the system 100 may still have the remaining part or parts of the status information stored in the data lake 128 should they be required.

By executing the executable instructions, the coordination system 108 writes to the distributed ledger 110 to update the status of the smart contract. In this regard, by executing the executable instructions the coordination system 108 determines that the current milestone or subprocess (corresponding to the executable instructions) has been completed and identifies a subsequent milestone or subprocess to be achieved. The coordination system 108 updates the current status of the smart contract 124 on the distributed ledger by writing to the distributed ledger to store a new current status corresponding to the subsequent milestone or subprocess to be achieved. Accordingly, when the distributed ledger 110 is next queried for the current status of the smart contract, the updated current status will be returned.

Hence, while the executable instructions are retrieved and executed in accordance with smart contracts 124 stored on the distributed ledger, the executable instructions themselves are stored in the data storage location 114, rather than the distributed ledger 110. This means that the logic defined by the executable instructions does not need to be stored and maintained by the distributed ledger 110, which may reduce the computational resources required by the distributed ledger. Furthermore, storing the executable instructions in the data storage location 114 while the smart contracts 124 are stored on the distributed ledger 110 may make it easier to edit the executable instructions when required.

In accordance with embodiments of the method, it may be possible for the coordination system 108 to appropriately process and store received status information that relates to multiple processes and therefore smart contracts.

In some embodiments, executing the executable instructions further causes the processor 120 of the coordination system 108 to determine one or more commands or one or more computer executable instructions which are to be executed by the second apparatus 106 of the plurality of apparatuses 102, and transmit those commands or computer executable instructions to the second apparatus 106.

The commands or computer executable instructions allow the coordination system 108 to control the operation of the second apparatus 106 to handle the biological material in a manner dictated by the executable instructions executed by the coordination system 108.

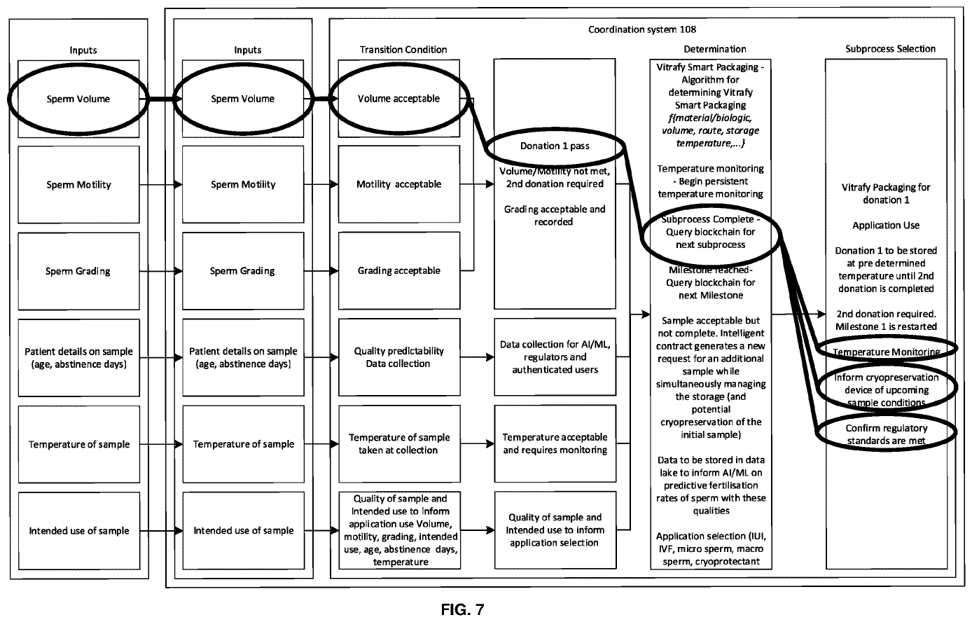

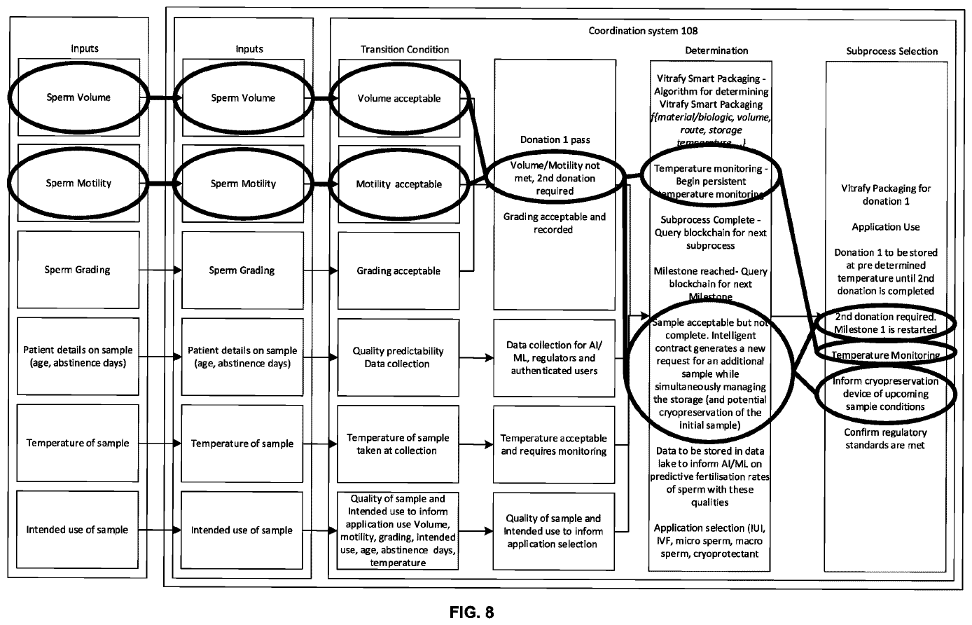

In some embodiments, the method 400 includes the coordination system 108 receiving historical status information and historical identification information for the biological material, as shown in FIGS. 5 and 6 at steps 502 and 602. The historical status information and historical identification information are received from at least one of the first apparatus 104 and the packaging device 126. As described hereinabove, the coordination system 108, apparatuses 102 and packaging device 126 may be connected in a mesh network. When status and identification information is transmitted from an apparatus 104, 106 or packaging device 126 to the coordination system 108 through the mesh, the status and identification information may be transmitted to the coordination system 108 through multiple communication pathways. Hence, the coordination system 108 may receive status and identification information multiple times.

If the coordination system 108 has previously received status and identification information corresponding to the historical status and identification information received at step 502 (or 602), then at step 504 the coordination system 108 determines whether the previously received status information should be updated with the historical identification information. This determination is made by comparing the historical status and identification information with the previously received status and identification information. If, based on the comparison, the coordination system 108 determines that the status and identification information has already been received (i.e., it is consistent with the previously received status and identification information), then it is determined that the previously received status information should not be updated with the historical identification information. If, based on the comparison, the coordination system 108 determines there to be an inconsistency between the previously received status information and the historical identification information, then at step 506 the coordination system 108 updates the previously received status information by storing an additional entry on the distributed ledger 110 recording the historical status information and flagging the inconsistency.

The coordination system 108 also determines, using the historical identification information, whether the coordination system 108 has previously received status and identification information corresponding to the historical status and identification information from the first apparatus 104 and/or the packaging device 126. If, at step 604, the coordination system 108 determines that the historical status information has not been previously received, then at step 406 the coordination system 108 stores the historical status information on the distributed ledger 110.

Accordingly, through the receipt of historical status and identification information from the first apparatus 104 and/or the packaging device 126, the coordination system 108 may be able to identify any errors in previously received status information, which may have been stored on the distributed ledger 110. The coordination system 108 may also be able to identify any ‘missing’ status information which ought to have been previously received and stored on the distributed ledger 110, but has not. The coordination system 108 may also avoid storing of multiple copies of status and identification information where it is received multiple times.

### Subprocess Selection Process

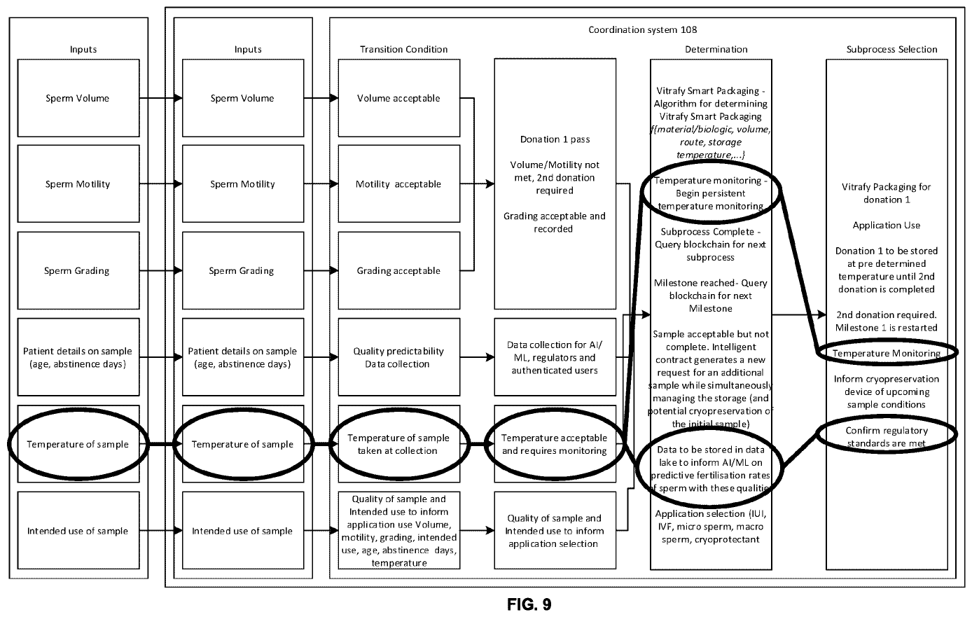

FIG. 7 shows an example of how the coordination system 108's receipt of status information leads to selection of a subsequent subprocess in an embodiment where the biological material is sperm collected from a patient for the purpose of ART.

In this example the coordination system 108 receives, from the input device 112, input in the form of status information being sperm volume of the collected sample. The input also includes the identification information (such as a smart contract identifier) which allows the coordination system 108 to retrieve the relevant smart contract 126 that corresponds to the current patient and ART process, and the current state of that smart contract 126. In response to receiving the status information and identification information, the coordination system 108 uses the identification information to query the distributed ledger 110 to retrieve the smart contract 126 associated with the sperm collected from the patient, including the current status of the smart contract. Once retrieved, the coordination system 108 uses the current status to query the data storage location 114 to retrieve executable instructions. The retrieved executable instructions relate to the current status of the smart contract 126. The coordination system 108 executes the retrieved executable instructions to cause the processor to (at least) process the status information and store at least a portion of the status information to the distributed ledger 110 as follows.

By executing the executable instructions, the coordination system 108 validates the status information to determine whether the sperm volume passes a transition condition (i.e., threshold sperm volume). In this instance, the coordination system 108 determines that the transition condition has been met (i.e., there is sufficient sperm volume). The coordination system 108 subsequently determines that the current subprocess is complete, and queries the distributed ledger 110 to identify the next subprocess to be achieved (i.e., the new “current status” of the smart contract 126), which in this case includes multiple concurrent subprocesses identified as being initiating temperature monitoring of the collected sample contained within the packaging device 126 by determining appropriate commands or computer executable instructions and transmitting the commands or computer executable instructions to the packaging device 126, informing a relevant cryopreservation apparatus (belonging to the plurality of apparatuses 102) of the upcoming biological material and its characteristics, and confirming that regulatory standards are met.

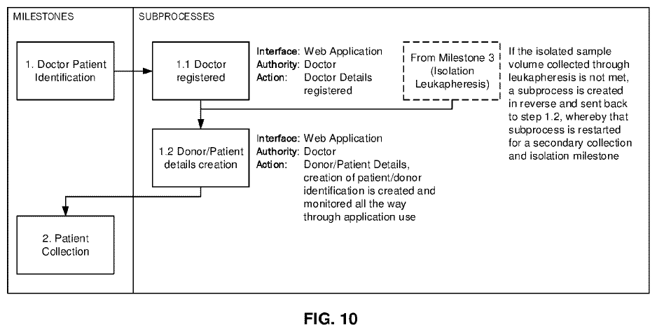

FIG. 8 shows another example of how the coordination system 108's receipt of status information leads to selection of a subsequent subprocess in an embodiment where the biological material is sperm collected from a patient for the purpose of ART.

In this example the coordination system 108 receives, from the input device 112, inputs in the form of status information being sperm volume and sperm motility of the collected sample. The input also includes the identification information (which may include a smart contract identifier or a patient identifier from which the coordination system 108 can derive the smart contract identifier) which allows the coordination system 108 to retrieve the relevant smart contract 126 that corresponds to the current patient and ART process, and the current state of that smart contract 126. In response to receiving the status information and identification information, the coordination system 108 uses the identification information to query the distributed ledger 110 to retrieve the smart contract 126 associated with the sperm collected from the patient, including the current status of the smart contract. Once retrieved, the coordination system 108 uses the current status to query the data storage location 114 to retrieve executable instructions. The retrieved executable instructions correspond to the current status of the smart contract 126. The coordination system 108 executes the retrieved executable instructions to cause the processor to (at least) process the status information and store to the distributed ledger as follows.

By executing the executable instructions, the coordination system 108 validates the inputs to determine whether the sperm volume and sperm motility pass respective transition conditions (i.e., threshold sperm volume and sperm motility requirements). In this instance, the coordination system 108 determines that the transition conditions have not been met (i.e., they both fail) and that a second donation is therefore required. The coordination system 108 subsequently initiates temperature monitoring of the sperm and generates a request for an additional sample. The coordination system 108 transmits the request to the input device 112, which provides the request to the user via the user interface 116. The coordination system 108 subsequently determines that since a second donation is required a previous milestone (corresponding to sample collection) must be restarted, and identifies the next subprocesses to be concurrently achieved as being: a second donation (restarting the previous milestone), temperature monitoring of the collected sample, and informing a relevant cryopreservation apparatus (belonging to the plurality of apparatuses 102) of the upcoming biological material and its characteristics.

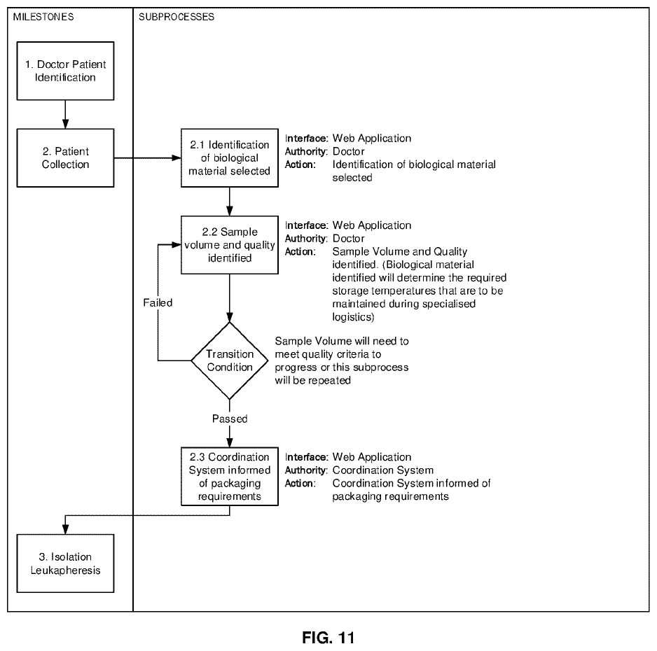

FIG. 9 shows a further example of how the coordination system 108's receipt of status information leads to selection of a subsequent subprocess in an embodiment where the biological material is sperm collected from a patient for the purpose of ART.

In this example the coordination system 108 receives, from the packaging device 126, input in the form of status information being temperature monitoring information for the collected sample. The input also includes the identification information (such as a smart contract identifier) which allows the coordination system 108 to retrieve the relevant smart contract 126 that corresponds to the current patient and ART process, and the current state of that smart contract 126. In response to receiving the status information and identification information, the coordination system 108 uses the identification information to query the distributed ledger 110 to retrieve the smart contract 126 associated with the sperm collected from the patient, including the current status of the smart contract. Once retrieved, the coordination system 108 uses the current status to query the data storage location 114 to retrieve executable instructions. The retrieved executable instructions correspond to the current status of the smart contract 126. The coordination system 108 executes the retrieved executable instructions to cause the processor to (at least) process the status information and store to the distributed ledger 110 as follows.

By executing the executable instructions, the coordination system 108 validates the status information to determine whether the temperature of the sample taken at collection passes a transition condition (i.e., acceptable temperature requirements) defined within the executable instructions. In this instance, the coordination system 108 determines that the temperature monitoring information is acceptable (i.e., the transition condition is passed). The coordination system 108 subsequently initiates temperature monitoring of the sperm contained within the packaging device 126 by determining appropriate commands or computer executable instructions and transmitting the commands or computer executable instructions to the packaging device 126, and causes the temperature monitoring information to be stored in the data lake 128. The coordination system 108 determines at least a portion of the received temperature monitoring information (or data derived from this information) that is to be stored on the distributed ledger 110, and causes that portion of the temperature monitoring information to be stored on the distributed ledger 110. The coordination system 108 subsequently identifies the next subprocesses to be concurrently achieved as being: continued temperature monitoring of the collected sample, and confirming that regulatory standards are met.

### Example 1: CAR-T (Chimeric Antigen Receptor T-Cell) Therapy

In a first example, the system and method described herein are used for controlling the handling of a biological material for the purposes of CAR-T therapy. In this example, the biological material includes peripheral blood mononuclear cells (‘PBMNCs’) which are genetically engineered into CAR-T cells.

In this example, the distributed ledger is a private blockchain, although in other examples a public blockchain may be used.

Initially, the user interface 116 receives user input requesting that a new process be initialised. The input indicates the required smart contract template 123 to be used to initialise a smart contract for the current process. The coordination system 108 receives the user input via the input device 112, and based on the user input is able to determine a template identifier corresponding to the required smart contract template 123. The coordination system 108 then uses the template identifier to send a request to the blockchain to initialise a smart contract 124. The request includes a smart contract identifier, which the coordination system 108 generates to be associated with the newly initialised smart contract and stored with the smart contract on the blockchain.

In this example, the user input is a selection made by a doctor indicating that they want to initiate a CAR-T therapy process. From the received user input, the coordination system 108 is able to identify the relevant template identifier as being that corresponding to the template 123 for a CAR-T therapy process. The coordination system 108 accordingly uses the CAR-T therapy template identifier to send a request to the blockchain to initialise a new smart contract 124 for CAR-T therapy. This includes generating a new smart contract identifier, which is sent to the blockchain with the request and is associated and stored with the newly initialised smart contract 124.

In this example, upon initialising the smart contract 124, the coordination system 108 updates the current status of the smart contract 124 to be subprocess 1.1 of milestone 1. Updating the current status includes submitting a write request to a gatekeeper component associated with the blockchain for a transaction associated with the current status of subprocess 1.1. The write request includes the smart contract identifier. In response to receiving the write request, the gatekeeper component executes a process for creating a new entity transaction for the data and submits the entity transaction to be committed to the blockchain in association with the relevant smart contract 124 for the present CAR-T therapy process. The coordination system 108 then receives confirmation from the gatekeeper that the current status has been stored to the blockchain.

### Milestone 1

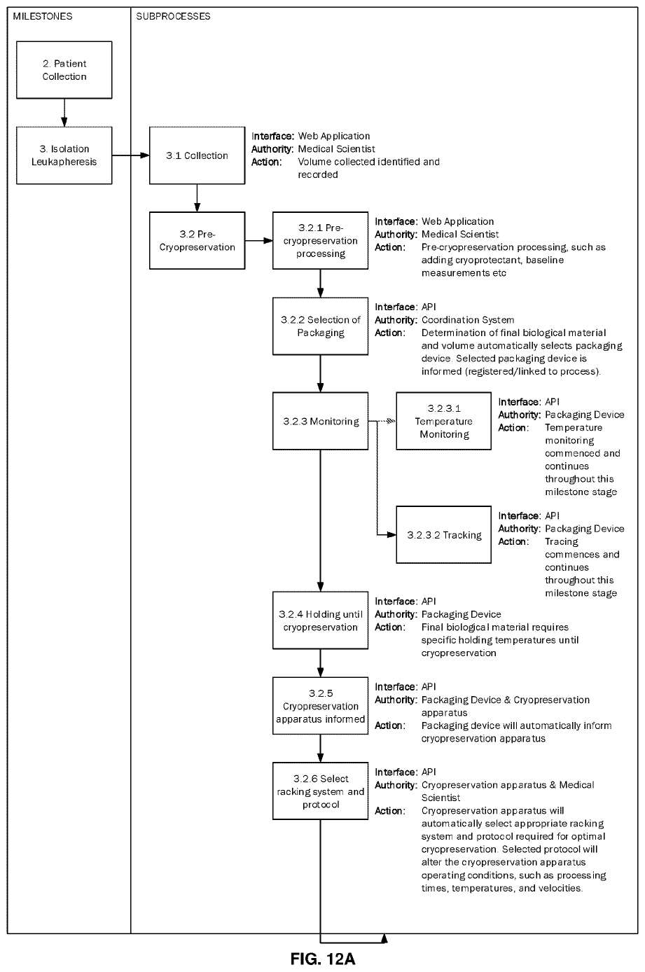

As shown in FIG. 10, a first milestone for this example is ‘Doctor Patient Identification’.

Based on the current status being subprocess 1.1, the coordination system 108 queries the data storage location 114 to retrieve executable instructions. In response, the coordination system 108 receives and executes executable instructions for subprocess 1.1, which in this example causes the processor 110 to do the following.

At subprocess 1.1, the coordination system 108 requests that the input device 112 instruct a doctor to provide doctor details (i.e., identifying the doctor that will perform the collection of the biological material from the patient). In this example the input device 112 is hosting a web application that communicates with the coordination system 108. In this example, the instruction is displayed on the user interface 116 associated with the input device 112, but the instructions may be provided to the doctor through additional or alternative means (e.g., audio instructions). The user interface 116 receives input of the doctor details from the doctor, and the input device 112 transmits the input doctor details to the coordination system 108. The coordination system 108 determines that the received doctor details are to be stored on the blockchain. Accordingly, the coordination system 108 stores the doctor details on the blockchain by submitting a write request to the gatekeeper component for a transaction with the doctor details. The write request includes the smart contract identifier. In response to receiving the write request, the gatekeeper component executes a process for creating a new entity transaction for the data and submits the entity transaction to be committed to the blockchain in association with the relevant smart contract 124 for the present CAR-T therapy process. The coordination system 108 receives confirmation from the gatekeeper that the data has been stored to the blockchain, and identifies that subprocess 1.1 is complete. As the smart contract 124 dictates that the next subprocess to be achieved is subprocess 1.2, the coordination system 108 writes to the distributed ledger to update the current status of the smart contract 124 to be subprocess 1.2 in the same manner described above for subprocess 1.1.

Based on the current status being subprocess 1.2, the coordination system 108 queries the data storage location 114 to retrieve executable instructions. In response, the coordination system 108 receives and executes executable instructions for subprocess 1.2, which in this example causes the processor 110 to do the following.

At subprocess 1.2, the coordination system 108 requests that the input device 112 instruct a doctor to provide patient details identifying the patient from which the biological material will be collected. The input device 112 provides the instruction by displaying it on the user interface 116. The user interface 116 receives input of the patient details from the doctor, and the input device 112 transmits the input patient details to the coordination system 108. The coordination system 108 determines that the received patient details are to be stored on the blockchain. Accordingly, the coordination system 108 stores the patient details on the blockchain (using the same steps described above at subprocess 1.1). Upon receiving confirmation from the gatekeeper that the data has been stored to the blockchain, the coordination system 108 identifies that subprocess 1.2 and milestone 1 are complete. As the smart contract 124 dictates that the next milestone is milestone 2, and the next subprocess to be achieved is subprocess 2.1, the coordination system 108 writes to the distributed ledger to update the current status of the smart contract 124 to be milestone 2 subprocess 2.1 in the same manner described above for subprocess 1.1.

### Milestone 2

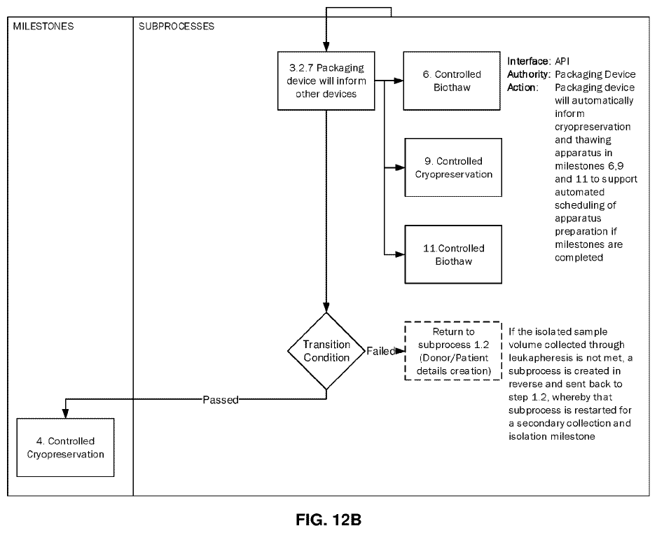

As shown in FIG. 11, a second milestone for this example is ‘Patient Collection’. At this milestone, the doctor collects a blood sample from the patient.

Based on the current status being subprocess 2.1, the coordination system 108 queries the data storage location 114 to retrieve executable instructions. In response, the coordination system 108 receives and executes executable instructions for subprocess 2.1, which in this example causes the processor 110 to do the following.

At subprocess 2.1, the coordination system 108 instructs the input device 112 to inform the doctor that they are required to input information identifying the type of the biological material and the relevant process or application for the biological material (e.g., through instructions displayed on the user interface 116). The user interface 116 receives input from the doctor identifying that the biological material is PBMNCs and the process is CAR-T therapy. The input device 112 transmits this input to the coordination system 108. Upon receipt of this input (which forms status information for the biological material), the coordination system 108 processes the status information identifying the type of the biological material and its application. The processing includes the coordination system 108 validating the status information to determine if the identified type of biological material and the application are valid for the smart contract 124. If the validation is successful, the coordination system 108 determines that the identified type of biological material and the application are to be stored on the blockchain. If the validation is unsuccessful, the coordination system 108 instructs the input device 112 to inform the user of the unsuccessful validation and request new input. The coordination system 108 may store a copy of the received status information in the data lake 128. Once processed, the coordination system 108 stores the status information to the blockchain (using the same steps described above at subprocess 1.1).

Upon receiving confirmation from the gatekeeper that the data has been stored to the blockchain, the coordination system 108 identifies that subprocess 2.1 is complete. As the smart contract 124 dictates that the next subprocess to be achieved is subprocess 2.2, the coordination system 108 writes to the blockchain to update the current status of the smart contract 124 to be subprocess 2.2 in the same manner described above for subprocess 1.1.

Based on the current subprocess being subprocess 2.2, the coordination system 108 queries the data storage location 114 to retrieve executable instructions. In response, the coordination system 108 receives and executes executable instructions for subprocess 2.2, which in this example causes the processor 110 to do the following.

At subprocess 2.2, the coordination system 108 instructs the input device 112 to inform the doctor that they are required to input status information of a volume of the collected blood sample and a quality of the collected blood sample (e.g., through instructions displayed on the user interface 116). The user interface 116 receives input from the doctor identifying the volume and quality of the collected blood sample. While in this example the status information is input by the doctor using the user interface 116, in other examples the status information may be received by the coordination system 108 from one or more of the apparatuses 102. The input device 112 transmits this input to the coordination system 108, which processes the input status information of volume and quality. The processing includes the coordination system 108 validating the status information to determine if the volume and quality of biological material and the application are valid for the smart contract 124—that is, whether the volume and quality meet predefined criteria (or “transition conditions”) defined by the executable instructions. If the validation is successful, the coordination system 108 determines that the received volume and quality of the biological material are to be stored on the blockchain. If the validation is unsuccessful, the coordination system 108 instructs the input device 112 to inform the doctor of the unsuccessful validation and request new input. The coordination system 108 may store a copy of the received status information in the data lake 128. Once processed, the coordination system 108 stores the status information to the blockchain (using the same steps described above at subprocess 1.1).

Upon receiving confirmation from the gatekeeper that the data has been stored to the blockchain, the coordination system 108 identifies that subprocess 2.2 is complete. As the smart contract 124 dictates that the next subprocess to be achieved is subprocess 2.3, the coordination system 108 writes to the blockchain to update the current status of the smart contract 124 to be subprocess 2.3 in the same manner described above for subprocess 1.1.

Based on the current status being subprocess 2.3, the coordination system 108 queries the data storage location 114 to retrieve executable instructions. In response, the coordination system 108 receives and executes executable instructions for subprocess 2.3, which in this example causes the processor 110 to do the following.

At subprocess 2.3 the coordination system 108 determines packaging requirements for the biological material. The coordination system 108 may make this determination based on status data relating to the biological material that is stored on the blockchain, which can be retrieved by querying the blockchain using the smart contract identifier. Relevant status information may include, for example, the sample volume and quality stored at subprocess 2.2.

Subsequently, the coordination system 108 identifies that subprocess 2.3 and milestone 2 are complete. As the smart contract 124 dictates that the next milestone and subprocess to be achieved are milestone 3 subprocess 3.1, the coordination system 108 writes to the distributed ledger to update the current status of the smart contract 124 to be milestone 3 subprocess 3.1 in the same manner described above for subprocess 1.1.

### Milestone 3

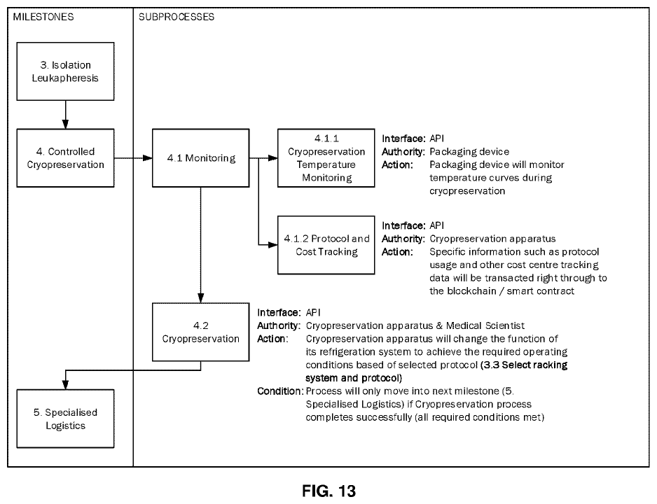

As shown in FIGS. 12A and 12B, a third milestone for this example is ‘Isolation Leukapheresis’. At this milestone, a medical scientist separates the blood sample using leukapheresis isolation technology to isolate and collect the biological material of interest, i.e., the PBMNCs.

Based on the current status being subprocess 3.1, the coordination system 108 queries the data storage location 114 to retrieve executable instructions. In response, the coordination system 108 receives and executes executable instructions for subprocess 3.1, which in this example causes the processor 110 to do the following.

At subprocess 3.1 the coordination system 108 instructs input device 112′ (which may be distinct from input device 112) to inform the medical scientist that they are required to input a volume of the isolated PBMNCs (e.g., through instructions displayed on a user interface 116′ associated with the input device 112′). The user interface 116′ receives input from the medical scientist identifying the volume collected. The input device 112′ transmits this input to the coordination system 108, which processes the volume collection details before storing them to the blockchain in the same manner as described above.

The coordination system 108 then receives confirmation from the gatekeeper that the data has been stored to the blockchain, and identifies that subprocess 3.1 is complete. As the smart contract 124 dictates that the next subprocess to be achieved is subprocess 3.2.1, the coordination system 108 writes to the blockchain to update the current status of the smart contract 124 to be subprocess 3.2.1 in the same manner described above for subprocess 1.1.

Based on the current status being subprocess 3.2.1, the coordination system 108 queries the data storage location 114 to retrieve executable instructions. In response, the coordination system 108 receives and executes executable instructions for subprocess 3.2.1, which in this example causes the processor 110 to do the following.

At subprocess 3.2.1, the coordination system 108 instructs the input device 112′ to inform the medical scientist that they are required to input status information relating to pre-cryopreservation processing that the medical scientist has performed in relation to the biological material using the input device 112′ (e.g., through instructions displayed on the user interface 116′). The pre-cryopreservation processing may include adding of cryoprotectant and conducting one or more baseline measurements (such as measurements of cell count, cell viability and/or cell functionality). The user interface 116′ receives input from the medical scientist identifying the pre-cryopreservation processing, and the input device 112′ transmits this input to the coordination system 108, which processes the pre-cryopreservation processing information before storing it to the blockchain in the same manner as described above.

The coordination system 108 then receives confirmation from the gatekeeper that the data has been stored to the blockchain, and identifies that subprocess 3.2.1 is complete. As the smart contract 124 dictates that the next subprocess to be achieved is subprocess 3.2.2, the coordination system 108 writes to the blockchain to update the current status of the smart contract 124 to be subprocess 3.2.2 in the same manner described above for subprocess 1.1.

Based on the current status being subprocess 3.2.2, the coordination system 108 queries the data storage location 114 to retrieve executable instructions. In response, the coordination system 108 receives and executes executable instructions for subprocess 3.2.2, which in this example causes the processor 110 to do the following.

At subprocess 3.2.2, the coordination system 108 selects an appropriate packaging device 126 for the biological material. According to the executable instructions, the selection is made based on at least the packaging requirements determined at subprocess 2.3. The coordination system 108 generates a packaging device identifier for the selected packaging device 126. The coordination system 108 stores the selection of the packaging device 126 (including the packaging device identifier) to the blockchain in the same manner as described above, and subsequently receives confirmation from the gatekeeper that the data has been stored to the blockchain.

The coordination system 108 informs the selected packaging device 126 that it has been selected for the current CAR-T therapy process. Informing the packaging device 126 may include the coordination system 108 transmitting a message to the packaging device 126. The message may include the smart contract identifier, or other information informing the packaging device that it has been selected, and optionally the therapy process for which it has been selected.

The coordination system 108 instructs the input device 112′ to inform the medical scientist that they are required to transfer the biological material (the isolated PBMNCs having undergone the pre-cryopreservation processing) into the selected packaging device 126 (e.g., through instructions displayed on the user interface 116′).

The coordination system 108 then identifies that subprocess 3.2.2 is complete. As the smart contract 124 dictates that the next subprocess to be achieved is subprocess 3.2.3, the coordination system 108 writes to the distributed ledger to update the current status of the smart contract 124 to be subprocess 3.2.3 in the same manner described above for subprocess 1.1.

Based on the current status being subprocess 3.2.3, the coordination system 108 queries the data storage location 114 to retrieve executable instructions. In response, the coordination system 108 receives and executes executable instructions for subprocess 3.2.3, which in this example causes the processor 110 to request the packaging device 126 to initiate monitoring by the sensors 308, and provide monitoring information to the coordination system 108. The monitoring information is generated from monitoring data captured by the sensors 308 of the packaging device 126. In this example, the monitoring information includes temperature monitoring information and location tracking information. The temperature monitoring information indicates the temperature of the biological material inside the packaging device 126, and is generated from monitoring data of a temperature sensor. The location tracking information indicates the location of the packaging device 126 (and therefore the biological material), and is generated from monitoring data of a location sensor (e.g., a GPS sensor). With the monitoring information, the coordination system 108 also receives identification information including the smart contract identifier. The identification information may also include a packaging device identifier for the packaging device 126.

The initiated monitoring persists throughout milestone 3, i.e., as subprocesses 3.2.4-3.2.7 are being carried out. When the coordination system 108 receives the monitoring information and identification information, the coordination system 108 uses the smart contract identifier to query the blockchain and retrieve the smart contract 124 for the current CAR-T therapy process, including the current status of that smart contract 124. Based on the retrieved smart contract 124 and the current status (which may be any of subprocesses 3.2.3-3.2.7), the coordination system 108 queries the data storage location 114 to retrieve the executable instructions for that subprocess, which when executed cause the processor 110 to process the received monitoring information. The processing includes the coordination system 108 validating the monitoring information to determine that the received temperature monitoring information and location tracking information meet predefined transition conditions. For example, the temperature monitoring information may be required to be within a certain range required to maintain the biological material within the packaging device 126. If the validation is unsuccessful, the coordination system 108 may send an alert to the medical scientist via the input device 112′ and user interface 116′. If the validation is successful, the coordination system 108 determines a portion of the monitoring information that is to be stored on the blockchain. For example, if the temperature monitoring information includes temperature measurements taken every second, the coordination system 108 may determine that one measurement every minute is to be stored on the blockchain. Once processed, the coordination system 108 stores the portion of the monitoring information to the blockchain (using the same steps described above at subprocess 1.1).

As described above, the coordination system 108 may store a copy of all received monitoring information in the data lake 128.

As described above, the packaging device 126 may locally store a copy of the monitoring information to the data store 306 of the packaging device. This local copy of the monitoring information can be received by the coordination system 108 at a later stage, e.g., for data integrity or data recovery purposes.

After initiating the monitoring, the coordination system 108 identifies that subprocess 3.2.3 is complete, notwithstanding that the initiated monitoring process will continue throughout the milestone. As the smart contract 124 dictates that the next subprocess to be achieved is subprocess 3.2.4, the coordination system 108 writes to the blockchain to update the current status of the smart contract 124 to be subprocess 3.2.4 in the same manner described above for subprocess 1.1.

Based on the current status being subprocess 3.2.4, the coordination system 108 queries the data storage location 114 to retrieve executable instructions. In response, the coordination system 108 receives and executes executable instructions for subprocess 3.2.4, which in this example causes the processor 110 to do the following.

At subprocess 3.2.4, the coordination system 108 determines that the biological material is to be held in the packaging device 126 at a holding temperature until cryopreservation. The holding temperature may include a range of acceptable temperatures. Accordingly, the coordination system 108 generates an alert if any of the temperature monitoring information received due to the monitoring initiated by subprocess 3.2.3 falls outside of the holding temperature range. The coordination system 108 then identifies that subprocess 3.2.4 is complete. As the smart contract 124 dictates that the next subprocess to be achieved is subprocess 3.2.5, the coordination system 108 writes to the blockchain to update the current status of the smart contract 124 to be subprocess 3.2.5 in the same manner described above.

Based on the current status being subprocess 3.2.5, the coordination system 108 queries the data storage location 114 to retrieve executable instructions. In response, the coordination system 108 receives and executes executable instructions for subprocess 3.2.5, which in this example causes the processor 110 to do the following.

At subprocess 3.2.5, the coordination system 108 is informed by at least one of the packaging device 126 and a first cryopreservation apparatus (belonging to the plurality of apparatuses 102) that the first cryopreservation apparatus has become aware of the packaging device 126. The first cryopreservation apparatus may be made aware of the packaging device 126 by detecting its proximity, e.g., using a LAN, WAN or PAN (such as by Wi-Fi, cellular network, RFID, Bluetooth or the like). In response to being informed, the coordination system 108 may inform the first cryopreservation apparatus that it will be used in the current CAR-T therapy process, linking it to the smart contract 124. This may include transmitting the smart contract identifier to the first cryopreservation apparatus. Additionally or alternatively, it may include transmitting status information relating to the biological material (which will be cryopreserved by the apparatus), such as the volume and quality information received at subprocess 2.2, to the first cryopreservation apparatus.

When the first cryopreservation device is made aware of the packaging device 126, it may also be informed of one or more of the following (directly from the packaging device 126 via the LAN, WAN or PAN, or via the coordination system 108): the type (e.g., including a size, shape and/or configuration) of the packaging device 126 selected at subprocess 3.2.2, the type of the biological material, and the pre-cryopreservation processing performed at 3.2.1 (e.g., the amount of cryoprotectant added).

In some embodiments, the coordination system 108 being informed of the first cryopreservation apparatus becoming aware of the packaging device 126 may result in the coordination system 108 allowing the packaging device 126 to be inserted into the first cryopreservation apparatus, e.g., by communicating with the first cryopreservation apparatus to unlock the apparatus.

The coordination system 108 then identifies that subprocess 3.2.5 is complete. As the smart contract 124 that the next subprocess to be achieved is subprocess 3.2.6, the coordination system 108 writes to the blockchain to update the current status of the smart contract 124 to be subprocess 3.2.6 in the same manner described above for subprocess 1.1.

Based on the current status being subprocess 3.2.6, the coordination system 108 queries the data storage location 114 to retrieve executable instructions. In response, the coordination system 108 receives and executes executable instructions for subprocess 3.2.6, which in this example causes the processor 110 to do the following.

At subprocess 3.2.6 the coordination system 108 receives status information in the form of a selected racking system and a cryopreservation protocol for cryopreservation of the biological material for the purpose of CAR-T therapy. The status information may be received via a secure API, or any other known communication mechanism. The racking system is configured to support one or more packaging devices inside the first cryopreservation apparatus to facilitate cryopreservation of biological material inside the packaging devices. Racking systems with different configurations can be used with the first cryopreservation apparatus to achieve different heat transfer rates, therefore, certain racking systems may be more suitable for use with certain packaging devices and biological materials. The cryopreservation protocol defines the cryopreservation settings of the first cryopreservation apparatus for optimally cryopreserving the biological material in the packaging device 126, including processing times, temperatures and heat exchange fluid velocities for the cryopreservation process.

The first cryopreservation apparatus may select the racking system based on one or more of: the type of the packaging device 126, the type of the biological material (i.e., CAR-T cells in this example) the volume of the biological material (e.g., number of cells) and the quality of the biological material. The cryopreservation apparatus may select the cryopreservation protocol based on one or more of: the type of the packaging device 126, the type of biological material (i.e., CAR-T cells in this example), the volume of the biological material (e.g., number of cells) and the quality of the biological material.

The coordination system 108 processes the received status information by validating it and determining that it is to be stored to the blockchain, and then writes to the blockchain to store the status information (using the same steps described above at subprocess 1.1). In embodiments where the first cryopreservation apparatus is configured to host at least one node 122 of the distributed ledger, the coordination system 108 may submit the write request to the gatekeeper component, where the gatekeeper component is on the first cryopreservation apparatus.

The coordination system 108 then identifies that subprocess 3.2.6 is complete. As the smart contract 124 dictates that the next subprocess to be completed is subprocess 3.2.7, the coordination system 108 writes to the blockchain to update the current status of the smart contract 124 to be subprocess 3.2.6 in the same manner described above for subprocess 1.1.

The coordination system 108 may request that the input device 112′ instruct the medical scientist to load the selected racking system into the first cryopreservation apparatus, e.g., by displaying instructions on the user interface 116′.

Alternatively, at subprocess 3.2.6 the coordination system 108 receives a request from the first cryopreservation apparatus via a secure API to obtain a selection of a racking system and a cryopreservation protocol for cryopreservation of the biological material for the purpose of CAR-T therapy.

The coordination system 108 processes the received request by confirming that the first cryopreservation apparatus is one of the plurality of apparatuses 102 and therefore authorised to interact with the coordination system 108, and confirming the current status of the smart contract by querying the blockchain using the smart contract identifier.

Once the coordination system 108 has processed the request, it invokes a query process on the blockchain via the gatekeeper. The gatekeeper executes the query on the blockchain to obtain the required information, being the selection of the racking system and the cryopreservation protocol for the current process. A blockchain query data result with the required information is returned from the blockchain to the coordination system 108 via the gatekeeper. The coordination system 108 processes the blockchain query data result and then transmits it to the first cryopreservation apparatus via the secure API.

In embodiments where the first cryopreservation apparatus is configured to host at least one node 122 of the distributed ledger, the coordination system 108 may direct the first cryopreservation apparatus to directly invoke the query process on the blockchain via the gatekeeper component on the first cryopreservation apparatus.

The coordination system 108 may request that the input device 112′ instruct the medical scientist to load the selected racking system into the first cryopreservation apparatus, e.g., by displaying instructions on the user interface 116′.

The coordination system 108 receives confirmation from the first cryopreservation apparatus that the selected racking system and cryopreservation protocol have been received. The coordination system 108 then identifies that subprocess 3.2.6 is complete. As the smart contract 124 dictates that the next subprocess to be completed is subprocess 3.2.7, the coordination system 108 writes to the blockchain to update the current status of the smart contract 124 to be subprocess 3.2.6 in the same manner described above for subprocess 1.1.

Based on the current status being subprocess 3.2.7, the coordination system 108 queries the data storage location 114 to retrieve executable instructions. In response, the coordination system 108 receives and executes executable instructions for subprocess 3.2.7, which in this example causes the processor 110 to do the following.

At subprocess 3.2.7, the coordination system 108 informs other relevant apparatuses of the plurality of apparatuses 102 that they will be required in future milestones associated with the CAR-T therapy process, linking them to the smart contract 124. In this example, a first thawing apparatus for milestone 6, a second cryopreservation apparatus for milestone 9 and a second thawing apparatus for milestone 11 are informed. Informing the relevant apparatuses may include transmitting the smart contract identifier for the smart contract 124 to each apparatus. Informing the relevant apparatuses may include transmitting status information relating to the biological material, such as the type (e.g., including a size, shape and/or configuration) of the packaging device 126 selected at subprocess 3.2.2, the type of the biological material, and the pre-cryopreservation processing performed at 3.2.1 (e.g., the amount of cryoprotectant added).

The coordination system 108 then identifies that subprocess 3.2.7 is complete.

The coordination system 108 determines whether milestone 3 is completed by determining whether the volume of the isolated PBMNCs satisfies a predetermined threshold (or, as shown in FIG. 9, a “transition condition”). The threshold is a minimum volume required for the CAR-T therapy process to proceed. For example, to treat an 80 kg patient for diffuse large B cell lymphoma, the threshold may be about 10×10⁸ cells. If the threshold is not met, then the coordination system 108 writes to the blockchain to update the current status of the smart contract to include subprocess 1.2 in order to prompt an additional iteration of milestones 2 and 3 and obtain additional biological material to allow the process to proceed. At the additional iteration of milestone 3, the threshold is adjusted to take into account any volumes collected at previous iterations of milestones 2 and 3.

If multiple samples are collected and isolated (due to the threshold not being satisfied as described above), then at the conclusion of milestone 3 the coordination system 108 requests that the input device 112′ instructs the medical scientist to consolidate all the isolated samples into a single packaging device 126 (e.g., by displaying the instruction on the user interface 116′).

Once the coordination system 108 identifies that subprocess 3.2.7 and milestone 3 are complete, as the smart contract 124 dictates that the next milestone and subprocess to be achieved are milestone 4 and subprocess 4.1, the coordination system 108 writes to the blockchain to update the current status of the smart contract 124 to be milestone 4 subprocess 4.1 in the same manner described above for subprocess 1.1 (the current status also including 1.2 if the coordination system 108 determines at milestone 3 that the volume of isolated PBMNCs does not satisfy the predetermined threshold).

### Milestone 4

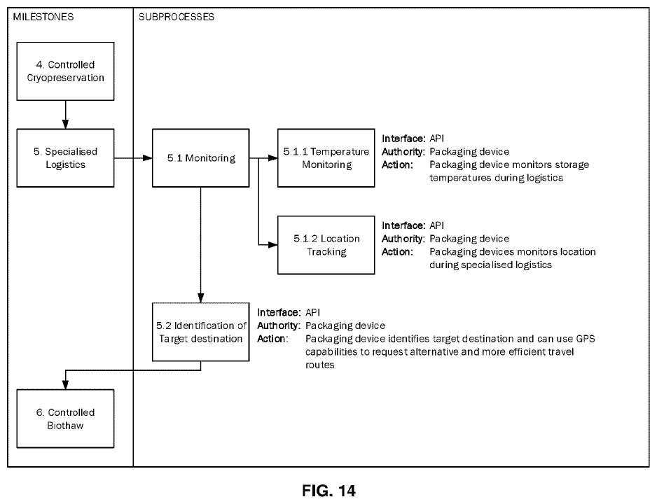

As shown in FIG. 13, a fourth milestone for this example is ‘Controlled Preservation’. At this milestone, a medical scientist places the packaging device 126 inside the first cryopreservation apparatus, which freezes the biological material in the packaging device 126 at a controlled rate.

Subprocess 4.1 is carried out in the same manner as described above for subprocess 3.2.3, but the request to initiate monitoring is sent to the first cryopreservation apparatus. The first cryopreservation apparatus generates the monitoring information from monitoring data captured by its sensors 208. At subprocess 4.1, the monitoring information may include temperature monitoring information (i.e., information about the temperature during cryopreservation) and operational monitoring information. The temperature monitoring information indicates the temperature of the biological material inside the packaging device 126, and is generated from monitoring data of a temperature sensor. The operational monitoring information indicates one or more operations carried out by the first cryopreservation apparatus, e.g., the velocity of heat exchange fluid. With the monitoring information, the coordination system 108 also receives identification information including the smart contract identifier. The identification information may also include an apparatus identifier for the first cryopreservation apparatus.

The initiated monitoring persists throughout milestone 4, i.e., as subprocess 4.2 is being carried out. When the coordination system 108 receives the monitoring information and identification information, the coordination system 108 uses the smart contract identifier to query the blockchain and retrieve the smart contract 124 for the current CAR-T therapy process, including the current status of that smart contract 124. Based on the retrieved smart contract 124 and the current status (which at that point may be subprocess 4.2), the coordination system 108 queries the data storage location 114 to retrieve the executable instructions for that subprocess, which when executed cause the processor 110 to process the received monitoring information. The processing includes the coordination system 108 validating the status information to determine that the received temperature monitoring information and operational monitoring information meet predefined transition conditions. For example, the operational monitoring information may be required to be within certain ranges to determine that there have not been any operational failures or other unforeseen events within the first cryopreservation apparatus. If the validation is unsuccessful, the coordination system 108 may send an alert to the medical scientist via the input device 112′ and user interface 116′. If the validation is successful, the coordination system 108 determines a portion of the monitoring information that is to be stored on the blockchain. For example, if the operational monitoring information includes monitoring information for a plurality of operations performed by the first cryopreservation apparatus, the coordination system 108 may determine that only the monitoring information for certain operations is to be stored on the blockchain. Once processed, the coordination system 108 stores the portion of the monitoring information to the blockchain (using the same steps described above at subprocess 1.1).

Processing the operational monitoring information may also include generating, based on the operational monitoring information, cost monitoring information indicating costs associated with operating the first cryopreservation apparatus to perform cryopreservation of the biological material; determining a portion of the cost monitoring information that is to be stored on the blockchain; and storing the portion of the cost monitoring information to the blockchain (using the same steps described above at subprocess 1.1).

After initiating the monitoring, the coordination system 108 identifies that subprocess 4.1 is complete, notwithstanding that the initiated monitoring will continue throughout the milestone. As the smart contract 124 dictates that the next subprocess to be achieved is subprocess 4.2, the coordination system 108 writes to the blockchain to update the current status of the smart contract 124 to be subprocess 4.2 in the same manner described above for subprocess 1.1.

Based on the current status being subprocess 4.2 the coordination system 108 queries the data storage location 114 to retrieve executable instructions. In response, the coordination system 108 receives and executes executable instructions for subprocess 4.2, which in this example causes the processor 110 to do the following.

At subprocess 4.2 the coordination system 108 controls the first cryopreservation apparatus to adjust its refrigeration system to achieve the required operating conditions in accordance with the cryopreservation protocol received at milestone 3.

The coordination system 108 identifies that subprocess 4.2 has been completed by determining, e.g., based on the operational monitoring information, that the operational conditions required by the cryopreservation protocol (selected at subprocess 3.2.6) have been satisfied by the first cryopreservation apparatus. As the smart contract 124 dictates that the next milestone and subprocess to be achieved are milestone 5 and subprocess 5.1, the coordination system 108 writes to the blockchain to update the current status of the smart contract 124 to be milestone 5 subprocess 5.1 in the same manner described above for subprocess 1.1.

### Milestone 5

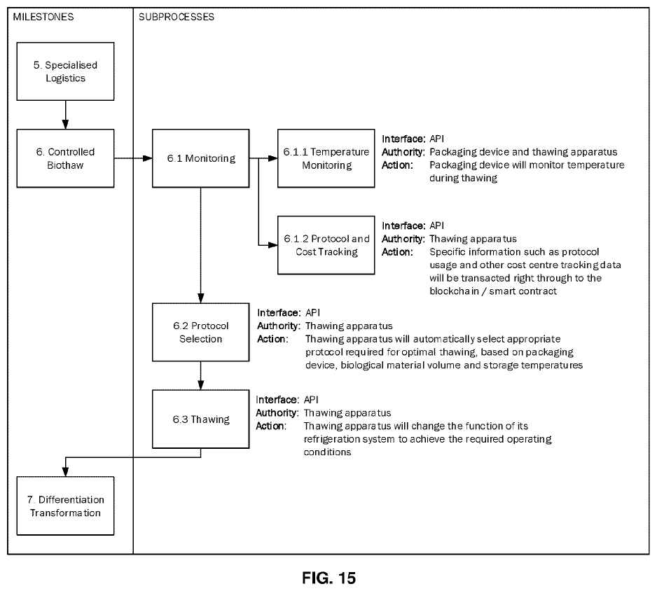

As shown in FIG. 14, a fifth milestone for this example is ‘Specialised Logistics’. At this milestone, the biological material inside the packaging device 126 is maintained in a controlled and monitored environment while in transit between the location of milestones 2-4 and the location of milestone 6.

Subprocess 5.1 is carried out in the same manner as described above for subprocess 3.2.3. The initiated monitoring persists throughout milestone 5, i.e., as subprocess 5.2 is being carried out. After initiating the monitoring, the coordination system 108 identifies that subprocess 5.1 is complete, notwithstanding that the initiated monitoring will continue throughout the milestone.

As the smart contract 124 dictates that the next subprocess to be achieved is subprocess 5.2, the coordination system 108 writes to the blockchain to update the current status of the smart contract 124 to be subprocess 5.2 in the same manner described above for subprocess 1.1.

Based on the current status being subprocess 5.2 the coordination system 108 queries the data storage location 114 to retrieve executable instructions. In response, the coordination system 108 receives and executes executable instructions for subprocess 5.2, which in this example causes the processor 110 to do the following.

At subprocess 5.2, the coordination system 108 determines a target destination of the biological material inside the packaging device 126. In this example the target destination corresponds to the location of the first thawing apparatus, which is pre-defined and stored on the blockchain. Based on the location tracking information received and stored due to the monitoring initiated at subprocess 5.2, the coordination system 108 may also determine a route (e.g., an optimal route) to the target destination.

Once the coordination system 108 has processed the request, it transmits the target destination to the secure API, which transmits the target destination to the packaging device 126.

The coordination system 108 receives confirmation from the packaging device 126 that the target destination has been received. The coordination system 108 then identifies that subprocess 5.2 and milestone 5 are complete. As the smart contract 124 dictates that the next milestone and subprocess to be achieved are milestone 6 and subprocess 6.1, the coordination system 108 writes to the blockchain to update the current status of the smart contract 124 to be milestone 6 subprocess 6.1 in the same manner described above for subprocess 1.1.

### Milestone 6

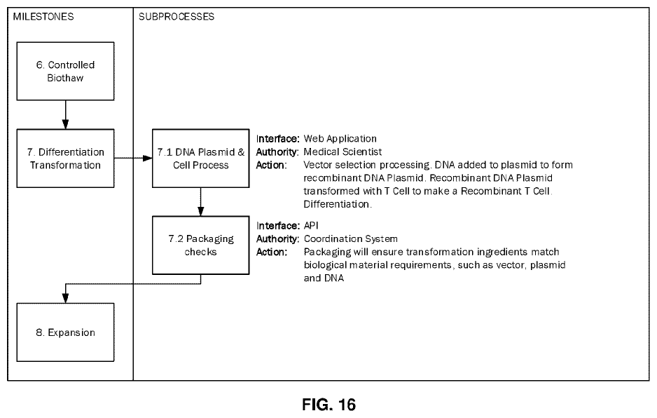

As shown in FIG. 15, a sixth milestone for this example is ‘Controlled Biothaw’. At this milestone, the cryopreserved biological material is thawed by the first thawing apparatus in preparation for transformation and expansion at milestone 7.

Subprocess 6.1 is carried out in the same manner as described above for subprocess 4.1, but the request to initiate monitoring is sent to the first thawing apparatus. The first thawing apparatus generates the monitoring information from monitoring data captured by its sensors 208. At subprocess 6.1, the monitoring information may include temperature monitoring information (i.e., to monitor the temperature during cryopreservation) and operational monitoring information. The temperature monitoring information indicates the temperature of the biological material inside the packaging device 126, and is generated from monitoring data of a temperature sensor. The operational monitoring information indicates one or more operations carried out by the first thawing apparatus, e.g., the thawing temperatures applied by the apparatus.

The initiated monitoring persists throughout milestone 6, i.e., as subprocesses 6.2 and 6.3 are being carried out. After initiating the monitoring, the coordination system 108 identifies that subprocess 6.1 is complete, notwithstanding that the initiated monitoring will continue throughout the milestone. As the smart contract 124 dictates that the next subprocess to be achieved is subprocess 6.2, the coordination system 108 writes to the blockchain to update the current status of the smart contract 124 to be subprocess 6.2 in the same manner described above for subprocess 1.1.

Based on the current status being subprocess 6.2 the coordination system 108 queries the data storage location 114 to retrieve executable instructions. In response, the coordination system 108 receives and executes executable instructions for subprocess 6.2, which in this example causes the processor 110 to do the following.

At subprocess 6.2 the coordination system 108 receives status information in the form of a selected thawing protocol for thawing the biological material in the packaging device. The thawing protocol defines the thawing settings of the first thawing apparatus, including warming speeds and temperatures for the thawing process.

The status information is received from the first thawing apparatus via a secure API. The first cryopreservation apparatus may select the thawing protocol based on one or more of: the type of the packaging device 126, the type of biological material (i.e., CAR-T cells in this example), the volume of the biological material (e.g., number of cells) and the temperatures at which the biological material has been stored at inside the packaging device 126 since cryopreservation.

The coordination system 108 processes the received status information by validating it and determining that it is to be stored to the blockchain, and then writes to the blockchain to store the status information (using the same steps described above at subprocess 1.1). The coordination system 108 then identifies that subprocess 6.2 is complete. As the smart contract 124 dictates that the next subprocess to be completed is subprocess 6.3, the coordination system 108 writes to the blockchain to update the current status of the smart contract 124 to be subprocess 6.3 in the same manner described above for subprocess 1.1.

Alternatively, at subprocess 6.2, the coordination system 108 receives a request from the first thawing apparatus via the secure API to obtain a selection of a thawing protocol for thawing the biological material in the packaging device 126, including warming speeds and temperatures for the thawing process.

The coordination system 108 processes the received request by confirming that the first thawing apparatus is one of the plurality of apparatuses 102 and therefore authorised to interact with the coordination system, and confirming the current status of the smart contract by querying the blockchain using the smart contract identifier.

Once the coordination system 108 has processed the request, it invokes a query process on the blockchain via the gatekeeper. The gatekeeper executes the query on the blockchain to obtain the required information, being the selection of the thawing protocol for the current process. A blockchain query data result with the required information is returned from the blockchain to the coordination system 108 via the gatekeeper. The coordination system 108 processes the blockchain query data result and then transmits it to the secure API. The secure API transmits the blockchain query result to the first thawing apparatus. The first thawing apparatus uses the received thawing protocol at subprocess 6.3 to thaw the biological material in the packaging device 126.

The coordination system 108 receives confirmation from the first thawing apparatus that the selected thawing protocol has been received. The coordination system 108 then identifies that subprocess 6.2 is complete. As the smart contract 124 dictates that the next subprocess to be completed is subprocess 6.3, the coordination system 108 writes to the blockchain to update the current status of the smart contract 124 to be subprocess 6.3 in the same manner described above for subprocess 1.1.

Based on the current status being subprocess 6.3, the coordination system 108 queries the data storage location 114 to retrieve executable instructions. In response, the coordination system 108 receives and executes executable instructions for subprocess 6.3, which in this example causes the processor 110 to do the following.

At subprocess 6.3 the coordination system 108 controls the first thawing apparatus to adjust its refrigeration system to achieve the required operating conditions in accordance with the thawing protocol received at subprocess 6.2. The coordination system 108 identifies that subprocess 6.3 has been completed by determining, e.g., based on the operational monitoring information that the operational conditions required by the thawing protocol (received at subprocess 6.2) have been satisfied by the first thawing apparatus. As the smart contract 124 dictates that the next milestone and subprocess to be achieved are milestone 7 and subprocess 7.1, the coordination system 108 writes to the blockchain to update the current status of the smart contract 124 to be milestone 7 subprocess 7.1 in the same manner described above for subprocess 1.1.

### Milestone 7

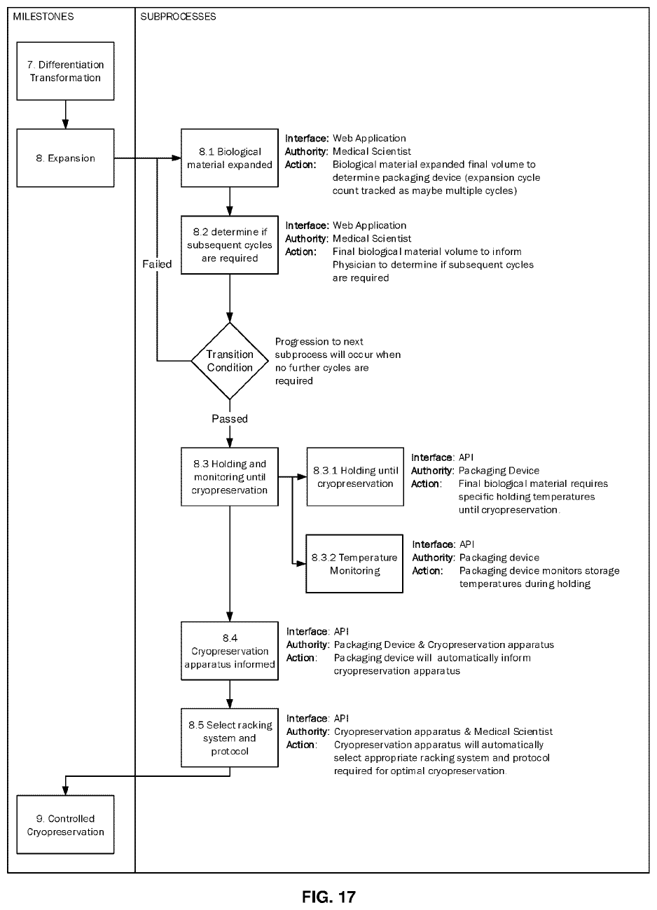

As shown in FIG. 16, a seventh milestone for this example is ‘Differentiation & Transformation’. At this milestone, the thawed biological material undergoes differentiation and transformation to generate CAR-T cells. This generally involves a medical scientist performing the following steps:
i. removing the biological material from the packaging device 126;
ii. selecting a vector and using the vector to remove a gene of interest from DNA containing cells (the removed DNA may be frozen and stored before being thawed when it is to be used);
iii. performing a DNA plasmid process, which may include isolation of plasmid and recombination of the plasmid with the gene of interest, to produce recombinant DNA plasmid;
iv. transformation of the recombinant DNA plasmid with the PBMNCs, e.g., through transfection, to create recombinant T cells (i.e., CAR-T cells); and
v. differentiation of the CAR-T cells, e.g., through cell sorting using a flow cytometer.

Based on the current status being subprocess 7.1 the coordination system 108 queries the data storage location 114 to retrieve executable instructions. In response, the coordination system 108 receives and executes executable instructions for subprocess 7.1, which in this example causes the processor 110 to do the following.

At subprocess 7.1, the coordination system 108 instructs input device 112″ (which may be distinct from input devices 112 and 112′) to inform the medical scientist performing the differentiation and transformation steps that they are required to input status information relating to the steps undertaken while generating the CAR-T cells. For example, the required information may include: the selected vector, identification of any media or cryoprotectants added or removed at each step, time taken to complete each step, total time taken to generate the CAR-T cells, cell count of biological material removed from packaging device 126, and viability count of biological material removed from packaging device 126. The instruction may be provided by displaying it on a user interface 116″ associated with the input device 112″.

The coordination system 108 receives, from the input device 112″, the status information input by the medical scientist via the user interface 116″. With this status information, the coordination system 108 also receives identification information including the smart contract identifier. Using the identification information, the coordination system 108 queries the blockchain to retrieve the smart contract 124 and the current status of subprocess 7.1. Based on the current status being subprocess 7.1, the coordination system 108 queries the data storage location 114 to retrieve executable instructions, which the coordination system 108 executes to process the status information. The processing includes validating the status information to determine if the input information meets predefined transition conditions. The transition conditions, for example, may be defined in the executable instructions based on one or more regulatory requirements for the process used to generate CAR-T cells used in CAR-T therapy. If the validation is unsuccessful, the coordination system 108 tags the invalid status information to identify that it did not pass validation and instructs the input device 112″ to inform the medical scientist of the unsuccessful validation. The medical scientist can then determine if the differentiation and transformation steps need to be repeated, or if the generated CAR-T cells cannot be used for CAR-T therapy (meaning that the current process must be terminated).

The processing also includes the coordination system 108 determining a portion of the status information that is to be stored on the blockchain (where the portion may include all of the status information). The coordination system 108 may store a copy of the received status information in the data lake 128. Once processed, the coordination system 108 stores the determined portion of the status information to the blockchain (using the same steps described above at subprocess 1.1).

The coordination system 108 then identifies that subprocess 7.1 is complete. As the smart contract 124 dictates that the next subprocess to be achieved is subprocess 7.2, the coordination system 108 writes to the distributed ledger to update the current status of the smart contract 124 to be subprocess 7.2 in the same manner described above for subprocess 1.1.

Based on the current status being subprocess 7.2, the coordination system 108 queries the data storage location 114 to retrieve executable instructions. In response, the coordination system 108 receives and executes executable instructions for subprocess 7.2, which in this example causes the processor 110 to do the following.

At subprocess 7.2, the coordination system 108 instructs input device 112″ to inform the medical scientist performing the differentiation and transformation steps that they are required to input status information relating to the final product biological material, being the generated CAR-T cells. For example, the required information may include (but is not limited to): confirmation that the CAR-T cells have the correct protein markers, cell count of the CAR-T cells, and viability count of the CAR-T cells. As for subprocess 7.1, the instruction may be provided by displaying it on a user interface 116″ associated with the input device 112″.

The coordination system 108 receives, from the input device 112″, the status information input by the medical scientist via the user interface 116″. With this status information, the coordination system 108 also receives identification information including the smart contract identifier. Using the identification information, the coordination system 108 queries the blockchain to retrieve the smart contract 124 and the current status of subprocess 7.1. Based on the current status being subprocess 7.1, the coordination system 108 queries the data storage location 114 to retrieve executable instructions, which the coordination system 108 executes to process the status information. The processing includes validating the status information to determine if the input information meets predefined transition conditions. The transition conditions, for example, may be defined in the executable instructions based on one or more regulatory requirements for CAR-T cells used in CAR-T therapy. If the validation is unsuccessful, the coordination system 108 tags the invalid status information to identify that it did not pass validation and instructs the input device 112″ to inform the user of the unsuccessful validation. The processing also includes the coordination system 108 determining a portion of the status information that is to be stored on the blockchain (where the portion may include all of the status information). The coordination system 108 may store a copy of the received status information in the data lake 128. Once processed, the coordination system 108 stores the determined portion of the status information to the blockchain (using the same steps described above at subprocess 1.1).

In some embodiments, at subprocess 7.2, executing the executable instructions for subprocess 7.2 also causes the coordination system 108 to determine packaging requirements and select an appropriate packaging device 126′ for the biological material (which is now in the form of differentiated CAR-T cells). This may be carried out similarly to the steps described at subprocesses 2.3 and 3.2.2 (in relation to determining packaging requirements and selecting packaging device 126). The coordination system 108 instructs the input device 112″ to inform the medical scientist that they are required to transfer the biological material (the differentiated CAR-T cells) into the selected packaging device 126′ (e.g., through instructions displayed on the user interface 116″). However, as further described hereinafter at subprocess 8.1, in some embodiments the selection of the packaging device 126′ may not occur until after the biological material has undergone expansion.

The coordination system 108 then identifies that milestone 7 and subprocess 7.2 are complete. As the smart contract 124 dictates that the next milestone and subprocess to be achieved are milestone 8 and subprocess 8.1, the coordination system 108 writes to the distributed ledger to update the current status of the smart contract 124 to be milestone 8 subprocess 8.1 in the same manner described above for subprocess 1.1.

### Milestone 8

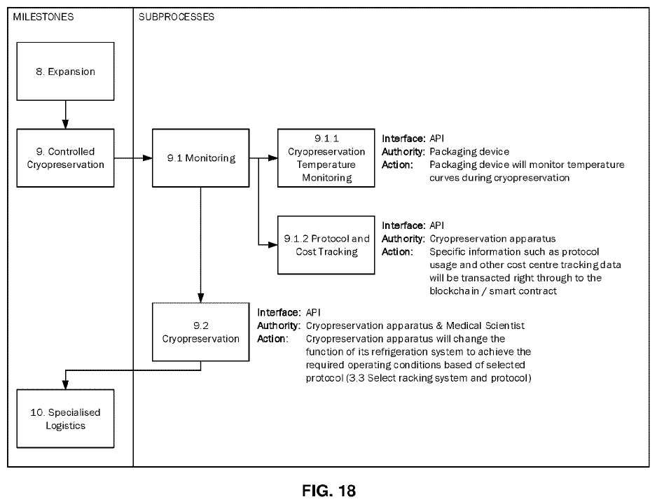

As shown in FIG. 17, an eighth milestone for this example is ‘Expansion’. At this milestone, the CAR-T cells are multiplied or ‘expanded’ in the laboratory by the medical scientist to a quantity that is sufficient for the intended use in CAR-T therapy.

Based on the current status being subprocess 8.1, the coordination system 108 queries the data storage location 114 to retrieve executable instructions. In response, the coordination system 108 receives and executes executable instructions for subprocess 8.1, which in this example causes the processor 110 to do the following.

At subprocess 8.1, the coordination system 108 requests that the input device 112″ instruct the medical scientist to expand the CAR-T cells and input status information relating to the expanded cells. For example, the required information may include a final volume of the expanded cells, a final cell count of the expanded cells, a quality and/or viability measurement for the expanded cells, and identification and volume of any media or cryoprotectant added to the expanded cells. The instructions may be provided to the medical scientist by displaying them on the user interface 116″.

The coordination system 108 receives status information from the input device 112″ (received from the medical scientist at the user interface 116″). With this status information, the coordination system 108 also receives identification information including the smart contract identifier. Using the identification information, the coordination system 108 queries the blockchain to retrieve the smart contract 124 and the current status of milestone 8 subprocess 8.1. Based on the current status being subprocess 8.1, the coordination system 108 queries the data storage location 114 to retrieve executable instructions, which the coordination system 108 executes to process the status information. The processing includes the coordination system 108 validating the status information to determine if the status information is valid for the smart contract 124—for example, whether the expansion volume satisfies predefined criteria (or “transition conditions”) defined by the executable instructions. If the validation is unsuccessful, the coordination system 108 tags the invalid status information to identify that it did not pass validation and instructs the input device 112″ to inform the user of the unsuccessful validation. The processing also includes the coordination system 108 determining a portion of the status information that is to be stored on the blockchain (where the portion may include all of the status information). The coordination system 108 may store a copy of the received expansion volume in the data lake 128. Once processed, the coordination system 108 stores the status information to the blockchain (using the same steps described above at subprocess 1.1).

In some embodiments, at subprocess 8.1, executing the executable instructions for subprocess 8.1 also causes the coordination system 108 to determine packaging requirements and select an appropriate packaging device 126′ for the biological material (which is now in the form of expanded CAR-T cells). This may be carried out similarly to the steps described at subprocesses 2.3 and 3.2.2 (in relation to determining packaging requirements and selecting packaging device 126). The coordination system 108 instructs the input device 112″ to inform the medical scientist that they are required to transfer the biological material (the expanded CAR-T cells) into the selected packaging device 126′ (e.g., through instructions displayed on the user interface 116″). However, as described hereinbefore at subprocess 7.2, in some embodiments the selection of the packaging device 126′ occurs before the biological material has undergone expansion. Whether this occurs before or after the expansion of the biological material may depend on the particular CAR-T therapy process that is being undertaken.

The coordination system 108 then identifies that subprocess 8.1 is complete. As the smart contract 124 dictates that the next subprocess to be achieved is subprocess 8.2, the coordination system 108 writes to the blockchain to update the current status of the smart contract 124 to be subprocess 8.2 in the same manner described above for subprocess 1.1.

Based on the current subprocess being subprocess 8.2, the coordination system 108 queries the data storage location 114 to retrieve executable instructions. In response, the coordination system 108 receives and executes executable instructions for subprocess 8.2, which in this example cause the processor 110 to do the following.

At subprocess 8.2, the coordination system 108 requests that the input device 112″ instruct the medical scientist to determine and input whether, based on the volume of the expanded CAR-T cells, the subsequent expansion cycles are required. The instructions may be provided to the medical scientist by displaying them on the user interface 116″.

The coordination system 108 receives status information from the input device 112″ in the form of user input from the medical scientist identifying whether further expansion cycles are required (received at the user interface 116″). With this status information, the coordination system 108 also receives identification information including the smart contract identifier. Using the identification information, the coordination system 108 queries the blockchain to retrieve the smart contract 124 and the current status of subprocess 8.2. Based on the current status being subprocess 8.2, the coordination system 108 queries the data storage location 114 to retrieve executable the instructions, which the coordination system 108 executes to process the status information. The processing includes the coordination system 108 validating the status information and determining that it is to be stored to the blockchain. The coordination system 108 stores the status information to the blockchain (using the same steps described above at subprocess 1.1). Upon receiving confirmation from the gatekeeper that the data has been stored to the blockchain, the coordination system 108 identifies that subprocess 8.2 is complete.

Following completion of subprocess 8.2, the current status of the smart contract 124 is updated based on a ‘Transition Condition’. If the input identifies that at least one further expansion cycle is required, then the ‘Transition Condition’ is failed and the smart contract 124 dictates that the next subprocess to be achieved is subprocess 8.1, which repeats as described above. If the input identifies that no further expansion cycles are required, then the ‘Transition Condition’ is passed and the smart contract 124 dictates that the next subprocess to be achieved is subprocess 8.3. Based on the outcome of the Transition Condition, the coordination system 108 writes to the blockchain to update the current status of the smart contract 124 to be either subprocess 8.1 or 8.3 in the same manner described above for subprocess 1.1.

If the current subprocess is subprocess 8.3, the coordination system 108 queries the data storage location 114 to retrieve executable instructions. In response, the coordination system 108 receives and executes executable instructions for subprocess 8.3, which in this example cause the processor 110 to do the following.

At subprocess 8.3, the coordination system 108 determines that the (expanded) biological material is to be held in the packaging device 126 at a holding temperature until it undergoes cryopreservation (8.3.1) and requests that the packaging device 126 initiate monitoring by the sensors 308 and provide monitoring information to the coordination system 108. The holding temperature may include a range of acceptable temperatures. The temperature monitoring information is generated in the same manner described at subprocess 3.2.3. The initiated monitoring persists throughout the remainder of milestone 8, i.e., as subprocesses 8.4 and 8.5 are being carried out, in the same manner described at subprocess 3.2.3, with the coordination system 108 storing the relevantly determined portion of the monitoring information to the blockchain (using the same steps described above at subprocess 1.1).

Accordingly, the coordination system 108 may generate an alert if any of the received temperature monitoring information falls outside of the holding temperature range. If the expansion process at subprocess 8.1 is not performed while the biological material is inside the packaging device 126, then the coordination system 108 may request that the input device 112″ instruct the medical scientist to return the expanded biological material to the packaging device 126 by displaying instructions on the user interface 116″.

The coordination system 108 then identifies that subprocess 8.3 is complete. As the smart contract 124 dictates that the next subprocess to be achieved is subprocess 8.4, the coordination system 108 writes to the blockchain to update the current status of the smart contract 124 to include subprocess 8.4 in the same manner described above.

Based on the current status being subprocess 8.4, the coordination system 108 queries the data storage location 114 to retrieve executable instructions. In response, the coordination system 108 receives and executes executable instructions for subprocess 8.4, which in this example causes the processor 110 to do the following.

At subprocess 8.4, the coordination system 108 is informed by at least one of the packaging device 126 and the second cryopreservation apparatus (belonging to the plurality of apparatuses 102) that the second cryopreservation apparatus has become aware of the packaging device 126. The second cryopreservation apparatus may be made aware of the packaging device 126 in the same manner described for subprocess 3.2.5.

The coordination system 108 then identifies that subprocess 8.4 is complete. As the smart contract 124 that the next subprocess to be achieved is subprocess 8.5, the coordination system 108 writes to the blockchain to update the current status of the smart contract 124 to be subprocess 8.5 in the same manner described above for subprocess 1.1.

Based on the current status being subprocess 8.5, the coordination system 108 queries the data storage location 114 to retrieve executable instructions. In response, the coordination system 108 receives and executes executable instructions for subprocess 8.5, in the same manner as described for subprocess 3.2.6, but with the coordination system 108 receiving status information in the form of a selected racking system and a cryopreservation protocol for cryopreservation by the second cryopreservation apparatus of the expanded biological material for the purpose of CAR-T therapy. Unlike at subprocess 3.2.6, the coordination system 108 identifies that milestone 8 and subprocess 8.5 are complete and identifies that the next milestone and subprocess to be achieved are milestone 9 and subprocess 9.1, and writes to the blockchain to update the current status of the smart contract 124 accordingly.

### Milestone 9

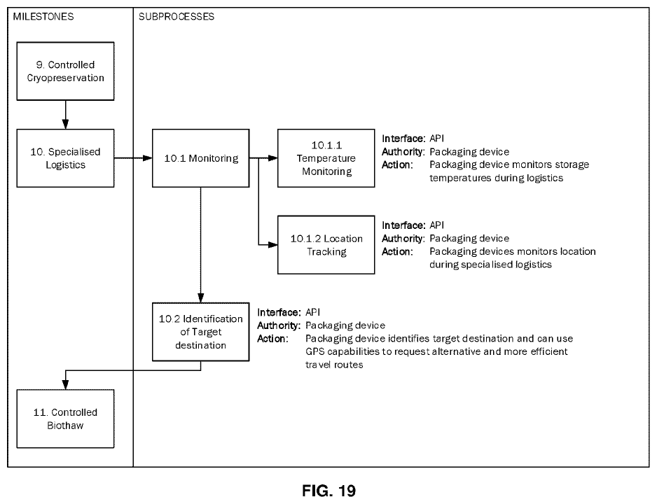

As shown in FIG. 18, a ninth milestone for this example is ‘Controlled Cryopreservation’. At this milestone, a medical scientist places the packaging device inside the second cryopreservation apparatus, which freezes the expanded CAR-T cells in the packaging device at a controlled rate.

Subprocess 9.1 corresponds to subprocess 4.1, as described above.

Subprocess 9.2 corresponds to subprocess 4.2, as described above, but for the second cryopreservation apparatus and the cryopreservation protocol received at milestone 8.

The coordination system 108 then identifies that milestone 9 and subprocess 9.2 are complete. As the smart contract 124 dictates that that the next milestone and subprocess to be achieved are milestone 10 and subprocess 10.1, the coordination system 108 writes to the blockchain to update the current status of the smart contract 124 to be milestone 10 subprocess 10.1 in the same manner described above for subprocess 1.1.

### Milestone 10

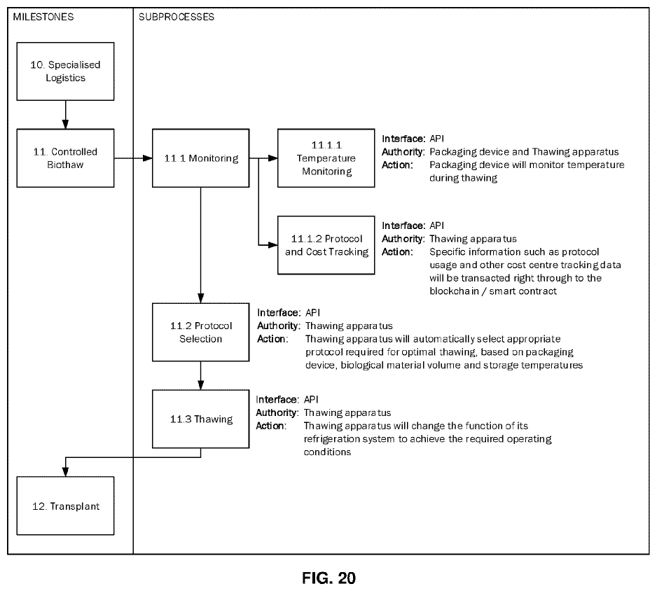

As shown in FIG. 19, a tenth milestone for this example is ‘Specialised Logistics’. At this milestone, the biological material inside the packaging device 126 is maintained in a controlled and monitored environment while in transit between the location of milestones 6-9 and the location of milestones 11-12.

Subprocess 10.1 corresponds to subprocess 5.1, as described above.

Subprocess 10.2 corresponds to subprocess 5.2, as described above.

The coordination system 108 then identifies that milestone 10 and subprocess 10.2 are complete. As the smart contract 124 dictates that the next milestone and subprocess to be achieved are milestone 11 and subprocess 11.1, the coordination system 108 writes to the blockchain to update the current status of the smart contract 124 to be milestone 11 subprocess 11.1 in the same manner described above for subprocess 1.1.

### Milestone 11

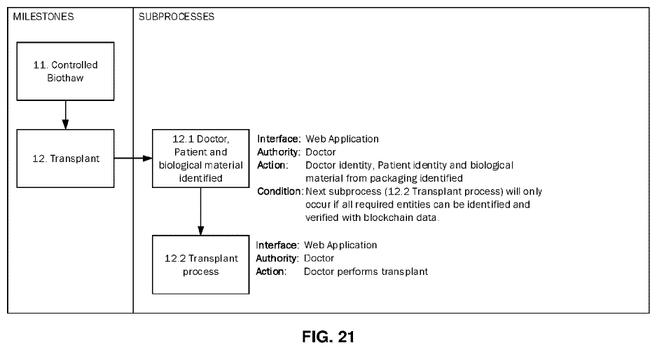

As shown in FIG. 20, an eleventh milestone for this example is ‘Controlled Biothaw’. At this milestone, the cryopreserved CAR-T cells are thawed by the second thawing apparatus in preparation for transplant at milestone 12.

Subprocess 11.1 corresponds to subprocess 6.1, as described above.

Subprocess 11.2 corresponds to subprocess 6.2, as described above, but for the second thawing apparatus.

Subprocess 11.3 corresponds to subprocess 6.3, as described above, but for the second thawing apparatus.

The coordination system 108 then identifies that milestone 11 and subprocess 11.3 are complete. As the smart contract 124 dictates that that the next milestone and subprocess to be achieved are milestone 12 and subprocess 12.1, the coordination system 108 writes to the blockchain to update the current status of the smart contract 124 to be milestone 12 subprocess 12.1 in the same manner described above for subprocess 1.1.

### Milestone 12

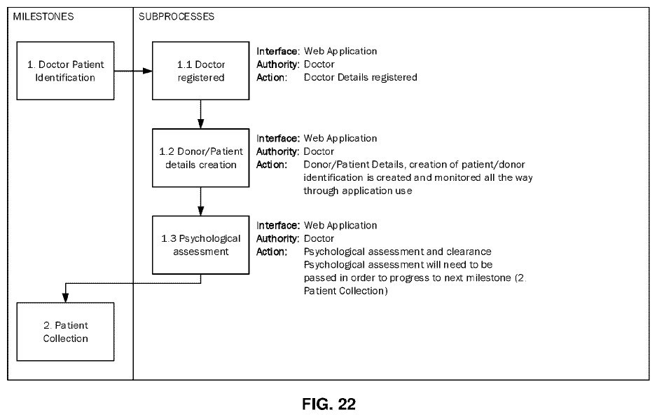

As shown in FIG. 21, a twelfth milestone for this example is ‘Transplant’. At this milestone, a medical professional such a doctor performs a transplant of the thawed CAR-T cells into the patient's blood stream.

Based on the current status being subprocess 12.1, the coordination system 108 queries the data storage location 114 to retrieve executable instructions. In response, the coordination system 108 receives and executes executable instructions for subprocess 12.1, which in this example causes the processor 110 to do the following.

At milestone 12.1, the coordination system 108 instructs the input device 112 to inform the doctor (who is to perform the transplant) that they are required to provide status information in the form of doctor details (identifying the doctor), patient details (identifying the transplant patient) and biological material details (identifying the biological material inside the packaging device 126). The input device 112 may provide the instructions to the doctor via the user interface 116. The details are input by the doctor using the user interface 116, and the input device 112 transmits the input status information to the coordination system 108. The coordination system 108 processes the received status information. The processing includes validating the status information to determine if the doctor details, patient details and biological material are valid for the smart contract 124. In this example, the validation may include querying the blockchain to check that the doctor details, patient details and biological material match the corresponding details stored on the blockchain. If the validation is successful, the coordination system 108 determines that the input doctor details, patient details and biological material are to be stored on the blockchain. If the validation is unsuccessful, the coordination system 108 instructs the input device to inform the user of the unsuccessful validation and request new input. The coordination system 108 may store a copy of the received status information in the data lake 128. Once processed, the coordination system 108 stores the status information to the blockchain (using the same steps described above at subprocess 1.1).

Upon receiving confirmation from the gatekeeper that the data has been stored to the blockchain, the coordination system 108 identifies that subprocess 12.1 is complete. As the smart contract 124 dictates that the next subprocess to be achieved is subprocess 12.2, the coordination system 108 writes to the blockchain to update the current status of the smart contract 124 to be subprocess 12.2 in the same manner described above for subprocess 1.1.

Based on the current status being subprocess 12.2, the coordination system 108 queries the data storage location 114 to retrieve executable instructions. In response, the coordination system 108 receives and executes executable instructions for subprocess 12.2, which in this example causes the processor 110 to do the following.

At subprocess 12.2, the coordination system 108 instructs the input device 112 to inform the doctor to perform the transplant, e.g., through a message displayed on the user interface 116. The coordination system 108 subsequently identifies that milestone 12, subprocess 12.2 and hence the entire CAR-T process defined by the smart contract 124 are complete.

### Example 2: Assisted Reproductive Therapy (ART)

In a second example, the system and method described herein are used for controlling the handling of a biological material for the purposes of ART therapy. In this example, the biological material includes sperm collected from a patient.

In this example, the distributed ledger is a private blockchain, although in other examples a public blockchain may be used.

Initially, the user interface 116 receives user input requesting that a new process be initialised. The input indicates the required smart contract template 123 to be used to initialise a smart contract for the current process. The coordination system 108 receives the user input via the input device 112, and based on the user input is able to determine a template identifier corresponding to the required smart contract template 123. The coordination system 108 then uses the template identifier to send a request to the blockchain to initialise a smart contract 124. The request includes a smart contract identifier, which the coordination system 108 generates to be associated with the newly initialised smart contract and stored with the smart contract on the blockchain.

In this example, the user input is a selection made by a doctor indicating that they want to initiate an ART process. From the received user input, the coordination system 108 is able to identify the relevant template identifier as being that corresponding to the template 123 for an ART process. The coordination system 108 accordingly uses the ART template identifier to send a request to the blockchain to initialise a new smart contract 124 for ART. This includes generating a new smart contract identifier, which is sent to the blockchain with the request and is associated and stored with the newly initialised smart contract 124.

Upon initialising the smart contract 124, the coordination system 108 updates the current status of the smart contract 124 to be subprocess 1.1 of milestone 1. Updating the current status includes submitting a write request to the gatekeeper component associated with the blockchain for a transaction associated with the current status of subprocess 1.1. The write request includes the smart contract identifier. In response to receiving the write request, the gatekeeper component executes a process for creating a new entity transaction for the data and submits the entity transaction to be committed to the blockchain in association with the relevant smart contract 124 for the present ART process. The coordination system 108 then receives confirmation from the gatekeeper that the current status has been stored to the blockchain.

### Milestone 1

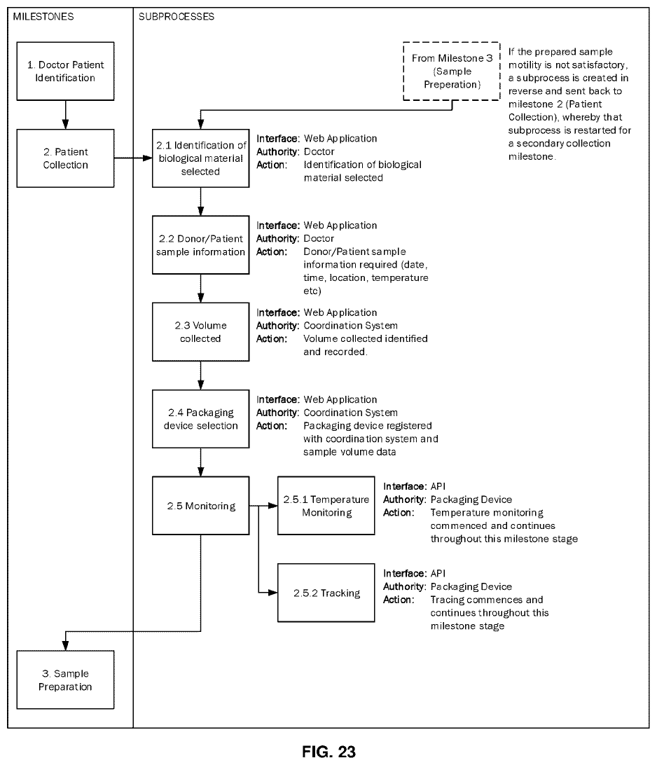

As shown in FIG. 22, a first milestone for this example is ‘Doctor Patient Identification’.

Subprocesses 1.1 and 1.2 are the same as subprocesses 1.1 and 1.2 in Example 1 (CAR-T Therapy) described above. However, in this example the patient may also be described as the donor.

At subprocess 1.3, the coordination system 108 requests that the input device 112 instruct the doctor to perform a psychological assessment of the patient and input results of the psychological assessment. The instruction may be provided to the doctor and/or other medical professional by displaying it on the user interface 116 associated with the input device 112.

The psychological assessment may take any appropriate form. For example, the psychological assessment may involve an assessment by the doctor and an assessment by an ART counsellor (e.g., an IVF counsellor). The assessment by the doctor may include determination of one or more of the following: patient age, amount of time the patient has been trying to conceive, patient genetic disposition, patient BMI, patient lifestyle, whether the patient is a smoker, patient consumption of alcohol, patient consumption of caffeinated beverages, patient physical activity, patient stressors, patient occupation and patient relationship status. The assessment by the counsellor may include determination of one or more of the following: patient informed consent for the ART procedure, patient understanding of risks/benefits/side effects/alternative treatments associated with the ART procedure, patient consent to de-identified patient information and treatment information being provided to one or more third party databases (e.g., Australia and New Zealand assisted reproductive database (ANZARD)), patient understanding of emotional implications of proceeding with the ART procedure (e.g., relating to adverse or undesired results), and patient understanding of potential personal and social implications for an individual born as a result of the ART procedure.

The results of the psychological assessment are input by the doctor and the counsellor using the user interface 116, and the input device 112 transmits this input to the coordination system 108. Upon receipt of this input (which forms status information for the biological material), the coordination system 108 processes the results of the psychological assessment. The processing includes the coordination system 108 validating the status information to determine if the results satisfy predefined transition conditions. The transition conditions, for example, may be defined in the executable instructions based on one or more regulatory requirements for ART procedures. If the validation is successful, the coordination system 108 determines that the results are to be stored on the blockchain. If the validation is unsuccessful, the coordination system 108 instructs the input device 112 to inform the user of the unsuccessful validation and request new input. The coordination system 108 may store a copy of the received status information in the data lake 128. Once processed, the coordination system 108 stores the status information to the blockchain (using the same steps described in Example 1).

Upon receiving confirmation from the gatekeeper that the data has been stored to the blockchain, the coordination system 108 identifies that milestone 1 and subprocess 1.3 are complete. As the smart contract 124 dictates that the next milestone and subprocess to be achieved are milestone 2 and subprocess 2.1, the coordination system 108 writes to the blockchain to update the current status of the smart contract 124 to be milestone 2 subprocess 2.1 in the same manner described above in Example 1.

### Milestone 2

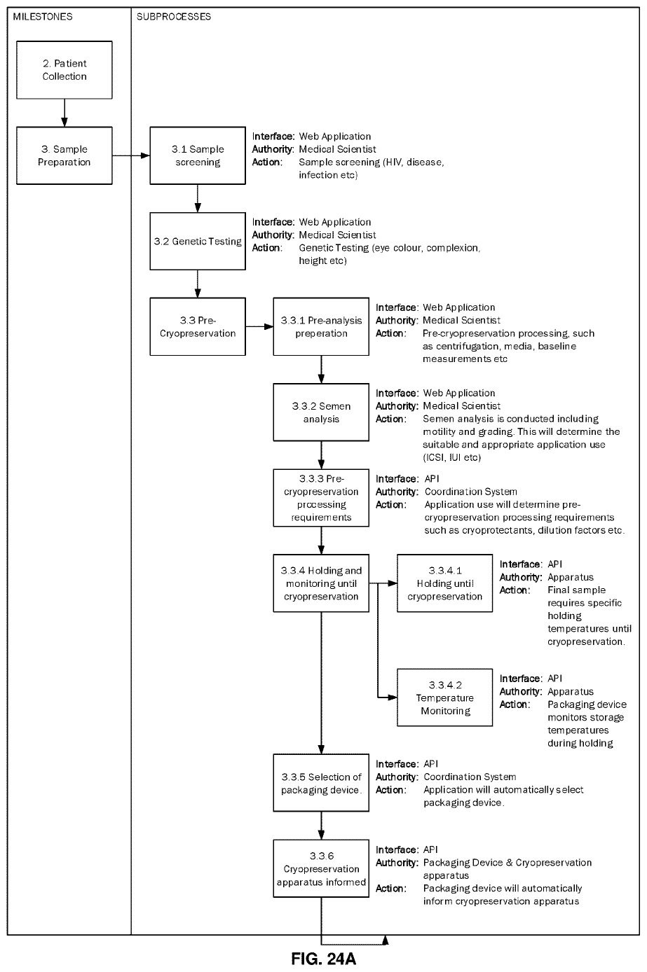

As shown in FIG. 23, a second milestone for this example is ‘Patient Collection’. At this milestone, the doctor obtains the biological material by collecting a semen sample from the patient.

Subprocess 2.1 is the same as subprocess 2.1 in Example 1, but the user interface 116 receives input from the doctor identifying that the biological material is semen and the process is ART.

At subprocess 2.2, the coordination system 108 instructs the input device 112 to inform the doctor that they are required to input status information relating to the collection of the semen sample (e.g., through instructions displayed on the user interface 116). In this example, the required information includes the date, time, location and ambient temperature at which the sample was collected. While in this example the information is input by the doctor using the user interface 116, in other examples the information may be automatically generated by the input device 112. The input device 112 transmits this input to the coordination system 108, which processes the input status information including the date, time, location and ambient temperature at the time of collection. The processing includes the coordination system 108 validating the status information to determine if the date, time, location and ambient temperature of the collection are valid for the smart contract 124—that is, whether the inputs meet predefined criteria (or “transition conditions”) defined by the executable instructions. If the validation is successful, the coordination system 108 determines that the received date, time, location and ambient temperature are to be stored on the blockchain. If the validation is unsuccessful, the coordination system 108 instructs the input device 112 to inform the doctor of the unsuccessful validation and request new input. The coordination system 108 may store a copy of the received status information in the data lake 128. Once processed, the coordination system 108 stores the status information to the blockchain (using the same steps described above in Example 1).

Upon receiving confirmation from the gatekeeper that the data has been stored to the blockchain, the coordination system 108 identifies that subprocess 2.2 is complete. As the smart contract 124 dictates that the next subprocess to be achieved is subprocess 2.3, the coordination system 108 writes to the blockchain to update the current status of the smart contract 124 to be subprocess 2.3 in the same manner described above in Example 1.

Based on the current status being subprocess 2.3, the coordination system 108 queries the data storage location 114 to retrieve executable instructions. In response, the coordination system 108 receives and executes executable instructions for subprocess 2.3, which in this example causes the processor 110 to do the following.

At subprocess 2.3, the coordination system 108 instructs the input device 112 to inform the doctor that they are required to collect a semen sample from the donor, and input status information of a volume of the collected semen sample (e.g., through instructions displayed on the user interface 116). The user interface 116 receives input from the doctor identifying the volume of the collected semen sample. While in this example the status information is input by the doctor using the user interface 116, in other examples the status information may be received by the coordination system 108 from one or more of the apparatuses 102 (e.g., an apparatus that measures the volume of the collected semen sample). The input device 112 transmits this input to the coordination system 108, which processes the input status information of volume. The processing includes the coordination system 108 validating the status information to determine if the volume of the biological material is valid for the smart contract 124—that is, whether the volume meets predefined criteria (or “transition conditions”) defined by the executable instructions. If the validation is successful, the coordination system 108 determines that the received volume is to be stored on the blockchain. If the validation is unsuccessful, the coordination system 108 instructs the input device 112 to inform the doctor of the unsuccessful validation and request new input. The coordination system 108 may store a copy of the received status information in the data lake 128. Once processed, the coordination system 108 stores the status information to the blockchain (using the same steps described above in Example 1.

Upon receiving confirmation from the gatekeeper that the data has been stored to the blockchain, the coordination system 108 identifies that subprocess 2.3 is complete. As the smart contract 124 dictates that the next subprocess to be achieved is subprocess 2.4, the coordination system 108 writes to the blockchain to update the current status of the smart contract 124 to be subprocess 2.4 in the same manner described above in Example 1.

Based on the current status being subprocess 2.4, the coordination system 108 queries the data storage location 114 to retrieve executable instructions. In response, the coordination system 108 receives and executes executable instructions for subprocess 2.4, which in this example causes the processor 110 to do the following.

At subprocess 2.4, the coordination system 108 selects an appropriate packaging device 126 for the biological material. According to the executable instructions, the selection may be made based on the volume of the biological material received at subprocess 2.3. The coordination system 108 generates a packaging device identifier for the selected packaging device 126. The coordination system 108 stores the selection of the packaging device 126 (including the packaging device identifier) to the blockchain in the same manner as described above, and subsequently receives confirmation from the gatekeeper that the data has been stored to the blockchain. The coordination system 108 informs the selected packaging device 126 that it has been selected for the current ART process. Informing the packaging device 126 may include the coordination system 108 transmitting the smart contract identifier to the packaging device 126.

The coordination system 108 instructs the input device 112 to inform the doctor that they are required to transfer the biological material (the collected sperm cells) into the selected packaging device 126 (e.g., through instructions displayed on the user interface 116).

The coordination system 108 then identifies that subprocess 2.4 is complete. As the smart contract 124 dictates that the next subprocess to be achieved is subprocess 2.5, the coordination system 108 writes to the distributed ledger to update the current status of the smart contract 124 to be subprocess 2.5 in the same manner described above in Example 1.

Based on the current status being subprocess 2.5, the coordination system 108 queries the data storage location 114 to retrieve executable instructions, which in this example causes the processor 110 to request the packaging device 126 to initiate monitoring by the sensors 308, and provide monitoring information to the coordination system 108. The monitoring information is generated from monitoring data captured by the sensors 308 of the packaging device 126. In this example, the monitoring information includes temperature monitoring information and location tracking information. The temperature monitoring information indicates the temperature of the biological material inside the packaging device 126, and is generated from monitoring data of a temperature sensor. The location tracking information indicates the location of the packaging device 126 (and therefore the biological material), and is generated from monitoring data of a location sensor (e.g., a GPS sensor). With the monitoring information, the coordination system 108 also receives identification information including the smart contract identifier. The identification information may also include the packaging device identifier for the packaging device 126.

The initiated monitoring persists until a termination condition defined in the executable instructions is satisfied. The termination condition may be, for example, a period of time (e.g., four hours), the current status of the smart contract 124 being a particular subprocess (e.g., subprocess 3.2), or the coordination system 108 receiving certain status information (e.g., receiving the required screening results at subprocess 3.1). When the coordination system 108 receives the monitoring information and identification information, the coordination system 108 uses the smart contract identifier to query the blockchain and retrieve the smart contract 124 for the current ART process, including the current status of that smart contract 124. Based on the retrieved smart contract 124 and the current status of subprocess 2.5, the coordination system 108 queries the data storage location 114 to retrieve the executable instructions for subprocess 2.5, which when executed cause the processor 110 to process the received monitoring information. The processing includes the coordination system 108 validating the monitoring information to determine that the received temperature monitoring information and location tracking information meet predefined transition conditions. For example, the temperature monitoring information may be required to be within a certain range required to maintain the viability of the biological material within the packaging device 126. If the validation is unsuccessful, the coordination system 108 may send an alert to the doctor via the input device 112 and user interface 116. If the validation is successful, the coordination system 108 determines a portion of the monitoring information that is to be stored on the blockchain. For example, if the temperature monitoring information includes temperature measurements taken every second, the coordination system 108 may determine that one measurement every minute is to be stored on the blockchain. Once processed, the coordination system 108 stores the portion of the monitoring information to the blockchain (using the same steps described above in Example 1).

As described above, the coordination system 108 may store a copy of all received monitoring information in the data lake 128.

As described above, the packaging device 126 may locally store a copy of the monitoring information to the data store 306 of the packaging device. This local copy of the monitoring information can be received by the coordination system 108 at a later stage, e.g., for data integrity or data recovery purposes.

After initiating the monitoring, the coordination system 108 identifies that subprocess 2.5 is complete, notwithstanding that the initiated monitoring process will continue after milestone 2 is complete until the termination condition is satisfied.

### Milestone 3

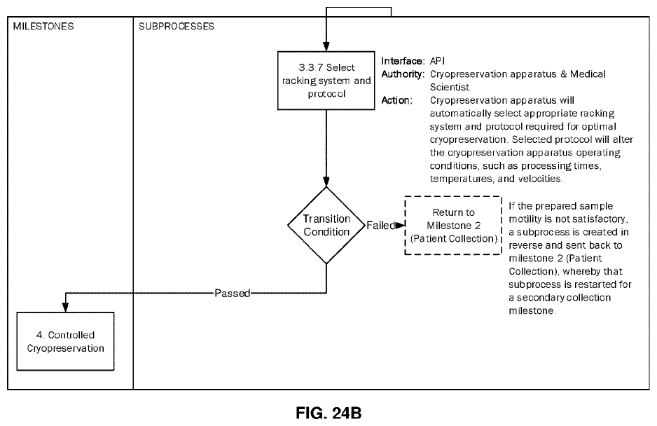

As shown in FIGS. 24A and 24B, a third milestone for this example is ‘Sample Preparation’. At this milestone, a medical scientist prepares the biological material (the collected semen sample) so that it is ready for cryopreservation.

Based on the current status being subprocess 3.1 the coordination system 108 queries the data storage location 114 to retrieve executable instructions. In response, the coordination system 108 receives and executes executable instructions for subprocess 3.1, which in this example causes the processor 110 to do the following.

At subprocess 3.1, the coordination system 108 instructs input device 112′ (which may be distinct to input device 112) to inform the medical scientist that they are required to input status information of screening results for the biological material (e.g., through instructions displayed on user interface 116′ associated with input device 112′). The required screening results may include results of one or more disease or infection tests performed on the biological material, such as results of a HIV test performed on the biological material. The user interface 116′ receives input from the medical scientist identifying the screening results. While in this example the screening results are input by the doctor using the user interface 116′, in other examples the screening results may be received by the coordination system 108 from one or more of the apparatuses 102 (e.g., one or more in vitro diagnostic apparatuses). The input device 112′ transmits this input to the coordination system 108, which processes the input status information. The processing includes the coordination system 108 validating the status information to determine that the received screening results meet predefined transition conditions. For example, certain screening results may be required for the semen sample to be capable of being used (e.g., due to regulatory requirements). If the validation is unsuccessful, the coordination system 108 may send an alert to the medical scientist via the input device 112′ and/or user interface 116′. If the validation is successful, the coordination system 108 determines at least a portion of the screening results that are to be stored on the blockchain. Once processed, the coordination system 108 stores the portion of the monitoring information (screening results) to the blockchain (using the same steps described above in Example 1).

The coordination system 108 then receives confirmation from the gatekeeper that the data has been stored to the blockchain, and identifies that subprocess 3.1 is complete. As the smart contract 124 dictates that the next subprocess to be achieved is subprocess 3.2, the coordination system 108 writes to the blockchain to update the current status of the smart contract 124 to be subprocess 3.2 in the same manner described above in Example 1.

Based on the current status being subprocess 3.2, the coordination system 108 queries the data storage location 114 to retrieve executable instructions. In response, the coordination system 108 receives and executes executable instructions for subprocess 3.2, which in this example causes the processor 110 to do the following.

At subprocess 3.2 the coordination system 108 instructs input device 112′ to inform the medical scientist that they are required to input status information relating to genetic testing that the medical scientist has performed in relation to the biological material using the input device 112′ (e.g., through instructions displayed on the user interface 116′). For example, the genetic testing may include testing for characteristics such as eye colour, complexion and/or height.

The coordination system 108 receives, from the input device 112′, the status information input by the medical scientist via the user interface 116′. With this status information, the coordination system 108 also receives identification information including the smart contract identifier. Using the identification information, the coordination system 108 queries the blockchain to retrieve the smart contract 124 and the current status of of the smart contract 124. Based on the retrieved current status being subprocess 3.2, the coordination system 108 queries the data storage location 114 to retrieve executable instructions, which the coordination system 108 executes to process the status information. The processing includes validating the status information to determine if the input information meets predefined transition conditions. The transition conditions, for example, may be defined in the executable instructions based on one or more predetermined requirements for the genetic testing results. If the validation is unsuccessful, the coordination system 108 tags the invalid status information to identify that it did not pass validation and instructs the input device 112″ to inform the medical scientist of the unsuccessful validation.

The processing also includes the coordination system 108 determining a portion of the status information that is to be stored on the blockchain (where the portion may include all of the status information). The coordination system 108 may store a copy of the received status information in the data lake 128. Once processed, the coordination system 108 stores the determined portion of the status information to the blockchain (using the same steps described above in Example 1).

The coordination system 108 then identifies that subprocess 3.2 is complete. As the smart contract 124 dictates that the next subprocess to be achieved is subprocess 3.3.1, the coordination system 108 writes to the distributed ledger to update the current status of the smart contract 124 to be subprocess 3.3.1 in the same manner described above for subprocess 1.1.

Based on the current status being subprocess 3.3.1, the coordination system 108 queries the data storage location 114 to retrieve executable instructions. In response, the coordination system 108 receives and executes executable instructions for subprocess 3.3.1, which in this example causes the processor 110 to do the following.

At subprocess 3.3.1, the coordination system 108 instructs the input device 112′ to inform the medical scientist that they are required to input status information relating to pre-analysis preparation that the medical scientist has performed in relation to the biological material to prepare it for subsequent analysis at subprocess 3.3.2 (e.g., through instructions displayed on the user interface 116′). The pre-analysis preparation may include processing such as centrifugation, addition of media, and conducting one or more baseline measurements (such as measurements of cell count, cell viability and/or cell functionality). The user interface 116′ receives input from the medical scientist identifying the pre-analysis preparation, and the input device 112′ transmits this input to the coordination system 108, which processes the pre-analysis preparation information before storing it to the blockchain in the same manner as described above in Example 1.

The coordination system 108 then receives confirmation from the gatekeeper that the data has been stored to the blockchain, and identifies that subprocess 3.3.1 is complete. As the smart contract 124 dictates that the next subprocess to be achieved is subprocess 3.3.2, the coordination system 108 writes to the blockchain to update the current status of the smart contract 124 to be subprocess 3.3.2 in the same manner described above in Example 1.

Based on the current status being subprocess 3.3.2, the coordination system 108 queries the data storage location 114 to retrieve executable instructions. In response, the coordination system 108 receives and executes executable instructions for subprocess 3.3.2, which in this example causes the processor to do the following.

At subprocess 3.3.2 the coordination system 108 instructs input device 112′ to inform the medical scientist that they are required to input status information relating to analysis undertaken on the biological material and corresponding analysis results using the input device 112′ (e.g., through instructions displayed on the user interface 116′). For example, the analysis relating to the application use may include determination of the motility of the sperm cells and determination of the grading of the sperm cells.

The user interface 116′ receives input from the medical scientist identifying the analysis steps and results. Although in this example the input is received from the user interface 116′, in other examples the input may also be received from one or more apparatuses that undertake the analysis of the biological material, the one or more apparatuses belonging to the plurality of apparatuses 102. The input device 112′ transmits this input to the coordination system 108, which processes the input status information. The processing includes the coordination system 108 validating the analysis steps and results to determine if they are valid for the smart contract 124—that is, whether the steps and results meet predefined criteria (or “transition conditions”) defined by the executable instructions. If the validation is successful, the coordination system 108 determines at least a portion of the analysis steps and results that are to be stored on the blockchain. If the validation is unsuccessful, the coordination system 108 instructs the input device 112′ to inform the medical scientist of the unsuccessful validation and request new input. The coordination system 108 may store a copy of the received status information in the data lake 128. Once processed, the coordination system 108 stores the determined portion of the status information to the blockchain (using the same steps described above in Example 1).

Upon receiving confirmation from the gatekeeper that the data has been stored to the blockchain, the coordination system 108 identifies that subprocess 3.3.2 is complete. As the smart contract 124 dictates that the next subprocess to be achieved is subprocess 3.3.3, the coordination system 108 writes to the blockchain to update the current status of the smart contract 124 to be subprocess 3.3.3 in the same manner described above in Example 1.

Based on the current status being subprocess 3.3.3 the coordination system 108 queries the data storage location 114 to retrieve executable instructions. In response, the coordination system 108 receives and executes executable instructions for subprocess 3.3.3, which in this example causes the processor 110 to carry out the same steps as described for subprocess 3.2.1 in Example 1. When the coordination system 108 identifies that subprocess 3.3.3 is complete, the smart contract 124 dictates that the next subprocess to be achieved is subprocess 3.3.4, and therefore the coordination system 108 writes to the blockchain to update the current status of the smart contract 124 to be subprocess 3.3.4 in the same manner described above in Example 1.

Based on the current subprocess being subprocess 3.3.4, the coordination system 108 queries the data storage location 114 to retrieve executable instructions. In response, the coordination system 108 receives and executes executable instructions for subprocess 3.3.4, which in this example causes the processor 110 to carry out the same steps as described for subprocess 8.3 in Example 1. When the coordination system 108 identifies that subprocess 3.3.4 is complete, the smart contract 124 dictates that the next subprocess to be achieved is subprocess 3.3.5, and therefore the coordination system 108 writes to the blockchain to update the current status of the smart contract 124 to be subprocess 3.3.5 in the same manner described above in Example 1.

Based on the current status being subprocess 3.3.5, the coordination system 108 queries the data storage location 114 to retrieve executable instructions. In response, the coordination system 108 receives and executes executable instructions for subprocess 3.3.5, which in this example causes the processor 110 to do the following.

At subprocess 3.3.5, the coordination system 108 selects an appropriate packaging device 126′ for the biological material, which by this stage has undergone pre-cryopreservation processing. According to the executable instructions, the selection may be made based on the volume of the biological material received at subprocess 2.3 and/or the pre-cryopreservation processing steps received at subprocess 3.3.1. The coordination system 108 generates a packaging device identifier for the selected packaging device 126′. The coordination system 108 stores the selection of the packaging device 126′ (including the packaging device identifier) to the blockchain in the same manner as described above, and subsequently receives confirmation from the gatekeeper that the data has been stored to the blockchain. The coordination system 108 informs the selected packaging device 126′ that it has been selected for the current ART process. Informing the packaging device 126′ may include the coordination system 108 transmitting the smart contract identifier to the packaging device 126′. In this example packaging device 126′ is distinct from packaging device 126 selected at subprocess 2.4, but in some examples packaging device 126′ may be the same as packaging device 126.

The coordination system 108 instructs the input device 112′ to inform the doctor that they are required to transfer the biological material (the collected sperm cells) into the selected packaging device 126′ (e.g., through instructions displayed on the user interface 116′).

The coordination system 108 then identifies that subprocess 3.3.5 is complete. As the smart contract 124 dictates that the next subprocess to be achieved is subprocess 3.3.6, the coordination system 108 writes to the distributed ledger to update the current status of the smart contract 124 to be subprocess 3.3.6 in the same manner described above in Example 1.

Based on the current subprocess being subprocess 3.3.6, the coordination system 108 queries the data storage location 114 to retrieve executable instructions. In response, the coordination system 108 receives and executes executable instructions for subprocess 3.3.6, which in this example causes the processor 110 to carry out the same steps as described for subprocess 3.2.5 in Example 1. When the coordination system 108 identifies that subprocess 3.3.6 is complete, the smart contract 124 dictates that the next subprocess to be achieved is subprocess 3.3.7, and therefore the coordination system 108 writes to the blockchain to update the current status of the smart contract 124 to be subprocess 3.3.7 in the same manner described above in Example 1.

Based on the current subprocess being subprocess 3.3.7, the coordination system 108 queries the data storage location 114 to retrieve executable instructions. In response, the coordination system 108 receives and executes executable instructions for subprocess 3.3.7, which in this example causes the processor 110 to carry out the same steps as described for subprocess 3.2.6 in Example 1.

When the coordination system 108 identifies that milestone 3 and subprocess 3.3.7 is complete, the smart contract 124 dictates that the next milestone and subprocess to be achieved are milestone 4 and subprocess 4.1, and therefore the coordination system 108 writes to the blockchain to update the current status of the smart contract 124 to be milestone 4 subprocess 4.1 in the same manner described above in Example 1.

### Milestone 4

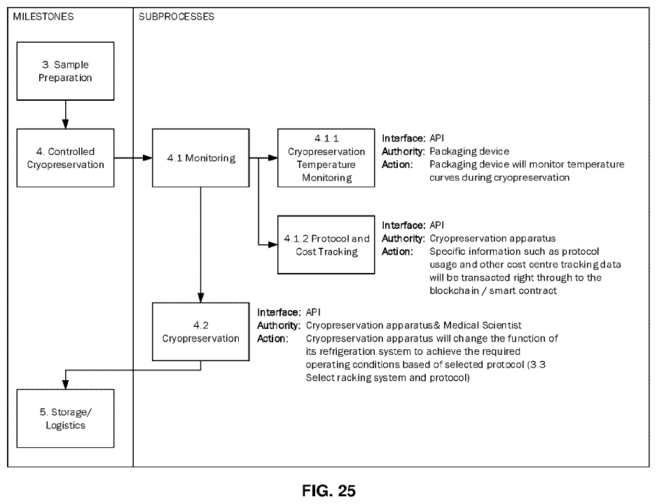

As shown in FIG. 25, a fourth milestone for this example is ‘Controlled Preservation’. At this milestone, a medical scientist places the packaging device 126 inside the cryopreservation apparatus, which freezes the biological material in the packaging device 126 at a controlled rate.

Subprocess 4.1 is carried out in the same manner as described above in subprocess 3.2.3 of Example 1, but the request to initiate monitoring is sent to the cryopreservation apparatus. The cryopreservation apparatus generates the monitoring information from monitoring data captured by its sensors 208. At subprocess 4.1, the monitoring information may include temperature monitoring information (i.e., information about the temperature during cryopreservation) and operational monitoring information. The temperature monitoring information indicates the temperature of the biological material inside the packaging device 126, and is generated from monitoring data of a temperature sensor. The operational monitoring information indicates one or more operations carried out by the cryopreservation apparatus, e.g., the velocity of heat exchange fluid. With the monitoring information, the coordination system 108 also receives identification information including the smart contract identifier. The identification information may also include an apparatus identifier that is associated with the cryopreservation apparatus.

The initiated monitoring persists throughout milestone 4, i.e., as subprocess 4.2 is being carried out. When the coordination system 108 receives the monitoring information and identification information, the coordination system 108 uses the smart contract identifier to query the blockchain and retrieve the smart contract 124 for the current ART process, including the current status of that smart contract 124. Based on the retrieved smart contract 124 and the current status (which at that point may be subprocess 4.2), the coordination system 108 queries the data storage location 114 to retrieve the executable instructions for that subprocess, which when executed cause the processor 110 to process the received monitoring information. The processing includes the coordination system 108 validating the status information to determine that the received temperature monitoring information and operational monitoring information meet predefined transition conditions. For example, the operational monitoring information may be required to be within certain ranges to determine that there have not been any operational failures or other unforeseen events within the cryopreservation apparatus. If the validation is unsuccessful, the coordination system 108 may send an alert to the medical scientist via the input device 112′ and user interface 116′. If the validation is successful, the coordination system 108 determines a portion of the monitoring information that is to be stored on the blockchain. For example, if the operational monitoring information includes monitoring information for a plurality of operations performed by the cryopreservation apparatus, the coordination system 108 may determine that only the monitoring information for certain operations is to be stored on the blockchain. Once processed, the coordination system 108 stores the portion of the monitoring information to the blockchain (using the same steps described above in Example 1).

Processing the operational monitoring information may also include generating, based on the operational monitoring information, cost monitoring information indicating costs associated with operating the cryopreservation apparatus to perform cryopreservation of the biological material; determining a portion of the cost monitoring information that is to be stored on the blockchain; and storing the portion of the cost monitoring information to the blockchain (using the same steps described above in Example 1).

After initiating the monitoring, the coordination system 108 identifies that subprocess 4.1 is complete, notwithstanding that the initiated monitoring will continue throughout the milestone. As the smart contract 124 dictates that the next subprocess to be achieved is subprocess 4.2, the coordination system 108 writes to the blockchain to update the current status of the smart contract 124 to be subprocess 4.2 in the same manner described above in Example 1.

Based on the current status being subprocess 4.2 the coordination system 108 queries the data storage location 114 to retrieve executable instructions. In response, the coordination system 108 receives and executes executable instructions for subprocess 4.2, which in this example causes the processor 110 to do the following.

At subprocess 4.2 the coordination system 108 controls the cryopreservation apparatus to adjust its refrigeration system to achieve the required operating conditions in accordance with the cryopreservation protocol received at milestone 3. It does this by determining one or more commands or one or more executable instructions, and transmitting the commands or executable instructions to the cryopreservation apparatus, which the cryopreservation apparatus executes to control the functions of its refrigeration system.

The coordination system 108 identifies that subprocess 4.2 has been completed by determining, e.g., based on the operational monitoring information, that the operational conditions required by the cryopreservation protocol (selected at subprocess 3.3.7) have been satisfied by the cryopreservation apparatus. As the smart contract 124 dictates that the next milestone and subprocess to be achieved are milestone 5 and subprocess 5.1, the coordination system 108 writes to the blockchain to update the current status of the smart contract 124 to be milestone 5 subprocess 5.1 in the same manner described above in Example 1.

### Milestone 5

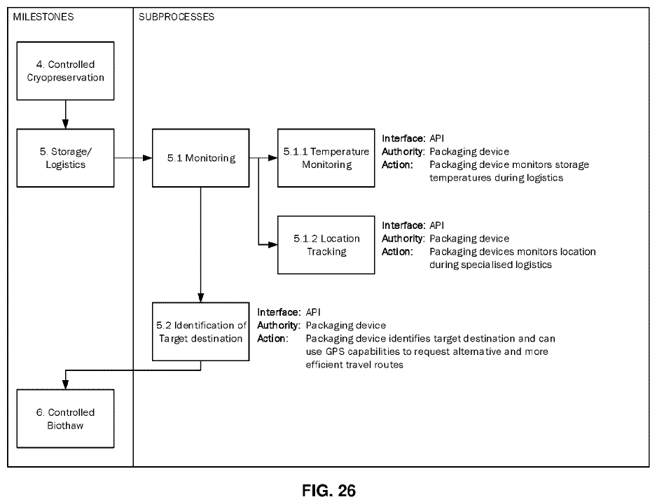

As shown in FIG. 26, a fifth milestone for this example is ‘Specialised Logistics’. At this milestone, the biological material inside the packaging device 126 is maintained in a controlled and monitored environment while in transit between the location of milestones 2-4 and the location of milestone 6.

Based on the current status being subprocess 5.1, the coordination system 108 queries the data storage location 114 to retrieve executable instructions. In response, the coordination system 108 receives and executes executable instructions for subprocess 5.1, which in this example causes the processor 110 to request the packaging device 126 to initiate monitoring by the sensors 308, and provide monitoring information to the coordination system 108. The monitoring information is generated from monitoring data captured by the sensors 308 of the packaging device 126. In this example, the monitoring information includes temperature monitoring information and location tracking information. The temperature monitoring information indicates the temperature of the biological material inside the packaging device 126, and is generated from monitoring data of a temperature sensor. The location tracking information indicates the location of the packaging device 126 (and therefore the biological material), and is generated from monitoring data of a location sensor (e.g., a GPS sensor). With the monitoring information, the coordination system 108 also receives identification information including the smart contract identifier. The identification information may also include a packaging device identifier for the packaging device 126.

The initiated monitoring persists throughout milestone 5, i.e., as subprocess 5.2 is being carried out. When the coordination system 108 receives the monitoring information and identification information, the coordination system 108 uses the smart contract identifier to query the blockchain and retrieve the smart contract 124 for the current ART process, including the current status of that smart contract 124. Based on the retrieved smart contract 124 and the current status (which may be subprocess 5.2), the coordination system 108 queries the data storage location 114 to retrieve the executable instructions for that subprocess, which when executed cause the processor 110 to process the received monitoring information. The processing includes the coordination system 108 validating the monitoring information to determine that the received temperature monitoring information and location tracking information meet predefined transition conditions. For example, the temperature monitoring information may be required to be within a certain range required to maintain the biological material within the packaging device 126. If the validation is unsuccessful, the coordination system 108 may send an alert to the medical scientist via the input device 112′ and user interface 116′. If the validation is successful, the coordination system 108 determines a portion of the monitoring information that is to be stored on the blockchain. For example, if the temperature monitoring information includes temperature measurements taken every second, the coordination system 108 may determine that one measurement every minute is to be stored on the blockchain. Once processed, the coordination system 108 stores the portion of the monitoring information to the blockchain (using the same steps described above in Example 1).

As described above, the coordination system 108 may store a copy of all received monitoring information in the data lake 128.

As described above, the packaging device 126 may locally store a copy of the monitoring information to the data store 306 of the packaging device. This local copy of the monitoring information can be received by the coordination system 108 at a later stage, e.g., for data integrity or data recovery purposes.

After initiating the monitoring, the coordination system 108 identifies that subprocess 5.1 is complete, notwithstanding that the initiated monitoring process will continue throughout the milestone. As the smart contract 124 dictates that the next subprocess to be achieved is subprocess 5.2, the coordination system 108 writes to the blockchain to update the current status of the smart contract 124 to be subprocess 5.2 in the same manner described above in Example 1.

Based on the current status being subprocess 5.2 the coordination system 108 queries the data storage location 114 to retrieve executable instructions. In response, the coordination system 108 receives and executes executable instructions for subprocess 5.2, which in this example causes the processor 110 to do the following.

At subprocess 5.2, the coordination system 108 determines a target destination of the biological material inside the packaging device 126. In this example the target destination corresponds to the location of the thawing apparatus, which is pre-defined and stored on the blockchain. Based on the location tracking information received and stored due to the monitoring initiated at subprocess 5.2, the coordination system 108 may also determine a route (e.g., an optimal route) to the target destination.

Once the coordination system 108 has processed the request, it transmits the target destination to the secure API, which transmits the target destination to the packaging device 126.

The coordination system 108 receives confirmation from the packaging device 126 that the target destination has been received. The coordination system 108 then identifies that subprocess 5.2 and milestone 5 are complete. As the smart contract 124 dictates that the next milestone and subprocess to be achieved are milestone 6 and subprocess 6.1, the coordination system 108 writes to the blockchain to update the current status of the smart contract 124 to be milestone 6 subprocess 6.1 in the same manner described above in Example 1.

### Milestone 6

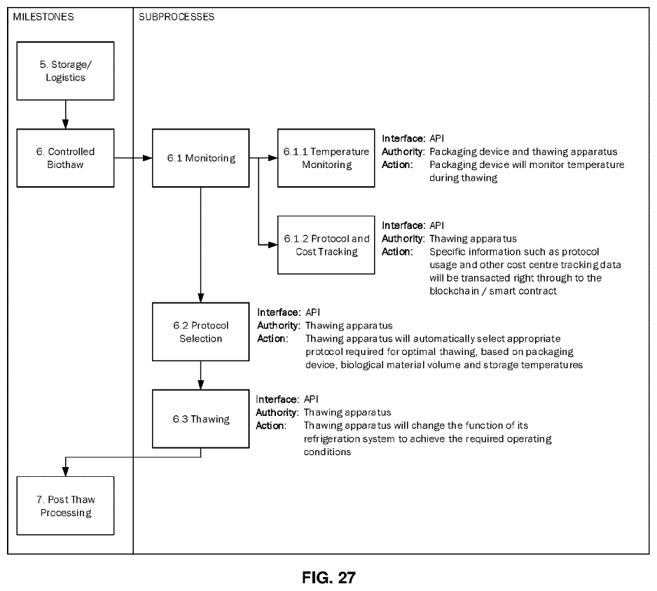

As shown in FIG. 27, a sixth milestone for this example is ‘Controlled Biothaw’. At this milestone, the cryopreserved biological material is thawed by the thawing apparatus in preparation for post thaw processing at milestone 7 and application use at milestone 8.

Subprocess 6.1 is carried out in the same manner as described above for subprocess 4.1, but the request to initiate monitoring is sent to the thawing apparatus. The thawing apparatus generates the monitoring information from monitoring data captured by its sensors 208. At subprocess 6.1, the monitoring information may include temperature monitoring information (i.e., to monitor the temperature during cryopreservation) and operational monitoring information. The temperature monitoring information indicates the temperature of the biological material inside the packaging device 126, and is generated from monitoring data of a temperature sensor. The operational monitoring information indicates one or more operations carried out by the thawing apparatus, e.g., the thawing temperatures applied by the apparatus.

The initiated monitoring persists throughout milestone 6, i.e., as subprocesses 6.2 and 6.3 are being carried out. After initiating the monitoring, the coordination system 108 identifies that subprocess 6.1 is complete, notwithstanding that the initiated monitoring will continue throughout the milestone. As the smart contract 124 dictates that the next subprocess to be achieved is subprocess 6.2, the coordination system 108 writes to the blockchain to update the current status of the smart contract 124 to be subprocess 6.2 in the same manner described above in Example 1.

Based on the current status being subprocess 6.2 the coordination system 108 queries the data storage location 114 to retrieve executable instructions. In response, the coordination system 108 receives and executes executable instructions for subprocess 6.2, which in this example causes the processor 110 to do the following.

At subprocess 6.2 the coordination system 108 receives status information in the form of a selected thawing protocol for thawing the biological material in the packaging device. The thawing protocol defines the thawing settings of the thawing apparatus, including warming speeds and temperatures for the thawing process.

The status information is received from the thawing apparatus via a secure API. The cryopreservation apparatus may select the thawing protocol based on one or more of: the type of the packaging device 126, the type of biological material (i.e., sperm cells in this example), the volume of the biological material (e.g., number of cells) and the temperatures at which the biological material has been stored at inside the packaging device 126 since cryopreservation.

The coordination system 108 processes the received status information by validating it and determining that it is to be stored to the blockchain, and then writes to the blockchain to store the status information (using the same steps described above at subprocess 1.1). The coordination system 108 then identifies that subprocess 6.2 is complete. As the smart contract 124 dictates that the next subprocess to be completed is subprocess 6.3, the coordination system 108 writes to the blockchain to update the current status of the smart contract 124 to be subprocess 6.3 in the same manner described above in Example 1.

Alternatively, at subprocess 6.2, the coordination system 108 receives a request from the thawing apparatus via the secure API to obtain a selection of a thawing protocol for thawing the biological material in the packaging device 126, including warming speeds and temperatures for the thawing process.

The coordination system 108 processes the received request by confirming that the thawing apparatus is one of the plurality of apparatuses 102 and therefore authorised to interact with the coordination system, and confirming the current status of the smart contract by querying the blockchain using the smart contract identifier.

Once the coordination system 108 has processed the request, it invokes a query process on the blockchain via the gatekeeper. The gatekeeper executes the query on the blockchain to obtain the required information, being the selection of the thawing protocol for the current process. A blockchain query data result with the required information is returned from the blockchain to the coordination system 108 via the gatekeeper. The coordination system 108 processes the blockchain query data result and then transmits it to the thawing apparatus via the secure API. The thawing apparatus uses the received thawing protocol at subprocess 6.3 to thaw the biological material in the packaging device 126.

The coordination system 108 receives confirmation from the thawing apparatus that the selected thawing protocol has been received. The coordination system 108 then identifies that subprocess 6.2 is complete. As the smart contract 124 dictates that the next subprocess to be completed is subprocess 6.3, the coordination system 108 writes to the blockchain to update the current status of the smart contract 124 to be subprocess 6.3 in the same manner described above in Example 1.

Based on the current status being subprocess 6.3, the coordination system 108 queries the data storage location 114 to retrieve executable instructions. In response, the coordination system 108 receives and executes executable instructions for subprocess 6.3, which in this example causes the processor 110 to do the following.

At subprocess 6.3 the coordination system 108 controls the thawing apparatus to adjust its refrigeration system to achieve the required operating conditions in accordance with the thawing protocol received at subprocess 6.2. The coordination system 108 identifies that subprocess 6.3 has been completed by determining, e.g., based on the operational monitoring information that the operational conditions required by the thawing protocol (received at subprocess 6.2) have been satisfied by the thawing apparatus. As the smart contract 124 dictates that the next milestone and subprocess to be achieved are milestone 7 and subprocess 7.1, the coordination system 108 writes to the blockchain to update the current status of the smart contract 124 to be milestone 7 subprocess 7.1 in the same manner described above in Example 1.

### Milestone 7

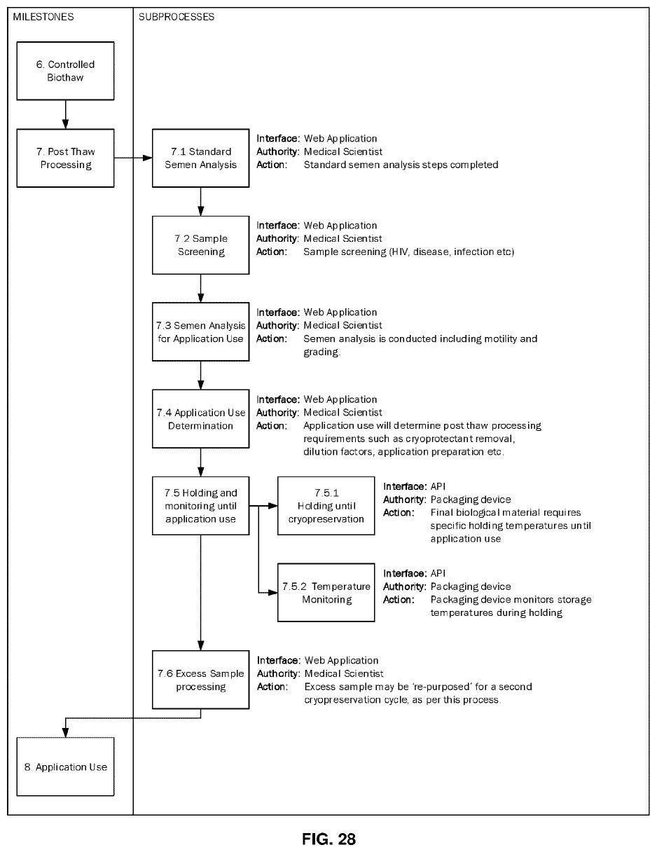

As shown in FIG. 28, a seventh milestone for this example is ‘Post Thaw Processing’. At this milestone, the thawed biological material undergoes analysis and screening before the thawed sperm cells are used in the ART procedure at milestone 8.

Based on the current status being subprocess 7.1 the coordination system 108 queries the data storage location 114 to retrieve executable instructions. In response, the coordination system 108 receives and executes executable instructions for subprocess 7.1, which in this example causes the processor 110 to do the following.

At subprocess 7.1, the coordination system 108 instructs input device 112″ (which may be distinct from input devices 112 and 112′) to inform the medical scientist performing the analysis and screening steps that they are required to input status information relating to the analysis steps undertaken on the thawed biological material, and the corresponding analysis results. For example, the required information may include: a measure of the motility of the sperm cells, a sperm count of the sperm cells, and/or results of a sperm DNA fragmentation test. The instruction may be provided by displaying it on a user interface 116″ associated with the input device 112″.

The user interface 116″ receives input from the medical scientist identifying the analysis steps and results. The input device 112″ transmits this input to the coordination system 108, which processes the input status information. The processing includes the coordination system 108 validating the analysis steps and results to determine if they are valid for the smart contract 124—that is, whether the steps and results meet predefined criteria (or “transition conditions”) defined by the executable instructions. If the validation is successful, the coordination system 108 determines at least a portion of the analysis steps and results that are to be stored on the blockchain. If the validation is unsuccessful, the coordination system 108 instructs the input device 112″ to inform the medical scientist of the unsuccessful validation and request new input. The coordination system 108 may store a copy of the received status information in the data lake 128. Once processed, the coordination system 108 stores the determined portion of the status information to the blockchain (using the same steps described above in Example 1).

Upon receiving confirmation from the gatekeeper that the data has been stored to the blockchain, the coordination system 108 identifies that subprocess 7.1 is complete. As the smart contract 124 dictates that the next subprocess to be achieved is subprocess 7.2, the coordination system 108 writes to the blockchain to update the current status of the smart contract 124 to be subprocess 7.2 in the same manner described above in Example 1.

Based on the current status being subprocess 7.2 the coordination system 108 queries the data storage location 114 to retrieve executable instructions. In response, the coordination system 108 receives and executes executable instructions for subprocess 7.2, which in this example causes the processor 110 to do the following.

At subprocess 7.2, the coordination system 108 instructs the input device 112″ to inform the medical scientist that they are required to input status information of screening results for the thawed biological material (e.g., through instructions displayed on the user interface 116″). Subprocess 7.2 is carried out in the same manner as subprocess 3.1 as described above, but in relation to the thawed biological material. Upon receiving confirmation from the gatekeeper that the data has been stored to the blockchain, the coordination system 108 identifies that subprocess 7.2 is complete. As the smart contract 124 dictates that the next subprocess to be achieved is subprocess 7.3, the coordination system 108 writes to the blockchain to update the current status of the smart contract 124 to be subprocess 7.3 in the same manner described above in Example 1.

Based on the current status being subprocess 7.3 the coordination system 108 queries the data storage location 114 to retrieve executable instructions. In response, the coordination system 108 receives and executes executable instructions for subprocess 7.3, which in this example causes the processor 110 to do the following.

At subprocess 7.3, the coordination system 108 instructs input device 112″ to inform the medical scientist that they are required to input status information relating to further analysis steps undertaken on the thawed biological material, the analysis relating to the application use at milestone 8, and the corresponding analysis results. For example, the analysis relating to the application use may include determination of the motility of the sperm cells and determination of the grading of the sperm cells. The instruction may be provided by displaying it on a user interface 116″ associated with the input device 112″.

The user interface 116″ receives input from the medical scientist identifying the analysis steps and results. The input device 112″ transmits this input to the coordination system 108, which processes the input status information. The processing includes the coordination system 108 validating the analysis steps and results to determine if they are valid for the smart contract 124—that is, whether the steps and results meet predefined criteria (or “transition conditions”) defined by the executable instructions. If the validation is successful, the coordination system 108 determines at least a portion of the analysis steps and results that are to be stored on the blockchain. If the validation is unsuccessful, the coordination system 108 instructs the input device 112″ to inform the medical scientist of the unsuccessful validation and request new input. The coordination system 108 may store a copy of the received status information in the data lake 128. Once processed, the coordination system 108 stores the determined portion of the status information to the blockchain (using the same steps described above in Example 1).

Upon receiving confirmation from the gatekeeper that the data has been stored to the blockchain, the coordination system 108 identifies that subprocess 7.3 is complete. As the smart contract 124 dictates that the next subprocess to be achieved is subprocess 7.4, the coordination system 108 writes to the blockchain to update the current status of the smart contract 124 to be subprocess 7.4 in the same manner described above in Example 1.

Based on the current status being subprocess 7.4 the coordination system 108 queries the data storage location 114 to retrieve executable instructions. In response, the coordination system 108 receives and executes executable instructions for subprocess 7.4, which in this example causes the processor 110 to do the following.

At subprocess 7.4, the coordination system 108 instructs input device 112″ to inform the medical scientist that they are required to input status information relating to post-thawing processing that the medical scientist has performed in relation to the biological material using the input device 112″ (e.g., e.g., through instructions displayed on the user interface 116″). The post-thawing processing may include any processing required before the thawed sperm cells can be used for the application use in milestone 8, such as removal of cryoprotectant, dilution of the sperm cells and any other preparation required for the specific ART procedure (e.g., IUI or ICSI) to be carried out with the sperm cells. The preparation for the specific ART procedure may include, for example, preparation of relevant instruments, dilution of the biological material to specific dilution factors, and measurement of relevant volumes of the biological material. The user interface 116″ receives input from the medical scientist identifying the post-thawing processing, and the input device 112″ transmits this input to the coordination system 108, which processes the post-thawing processing information before storing it to the blockchain in the same manner as described above in Example 1.

The coordination system 108 then receives confirmation from the gatekeeper that the data has been stored to the blockchain, and identifies that subprocess 7.4 is complete. As the smart contract 124 dictates that the next subprocess to be achieved is subprocess 7.5, the coordination system 108 writes to the blockchain to update the current status of the smart contract 124 to be subprocess 7.5 in the same manner described above in Example 1.

Based on the current status being subprocess 7.5 the coordination system 108 queries the data storage location 114 to retrieve executable instructions. In response, the coordination system 108 receives and executes executable instructions for subprocess 7.5, which in this example causes the processor 110 to do the following.

At subprocess 7.5, the coordination system 108 instructs input device 112″ to inform the medical scientist that they are required to reinsert the biological material (at this stage being the thawed sperm cells that have undergone post-thawing processing) into the packaging device 126. The coordination system 108 determines that the biological material is to be held in the packaging device 126 at a holding temperature until the biological material is required for the application use, and requests that the packaging device 126 initiate monitoring by the sensors 308 and provide monitoring information to the coordination system 108. The holding temperature may include a range of acceptable temperatures. The temperature monitoring information is generated in the same manner described for subprocess 3.2.3 in Example 1. The initiated monitoring persists throughout the remainder of milestone 7, i.e., as subprocess 7.6 is being carried out, in the same manner described for subprocess 3.2.3 in Example 1, with the coordination system 108 storing the relevantly determined portion of the monitoring information to the blockchain (using the same steps described above in Example 1).

Accordingly, the coordination system 108 generates an alert if any of the temperature monitoring information falls outside of the holding temperature range. The coordination system 108 then identifies that subprocess 7.5 is complete. As the smart contract 124 dictates that the next subprocess to be achieved is subprocess 7.6, the coordination system 108 writes to the blockchain to update the current status of the smart contract 124 to be subprocess 7.6 in the same manner described above in Example 1.

Based on the current status being subprocess 7.6 the coordination system 108 queries the data storage location 114 to retrieve executable instructions. In response, the coordination system 108 receives and executes executable instructions for subprocess 7.6, which in this example causes the processor 110 to do the following.

At subprocess 7.6, the coordination system 108 instructs input device 112″ to inform the medical scientist that they are required to input status information relating to whether an excess portion of the biological material is to undergo further cryopreservation, e.g., for subsequent further processing. The medical scientist will determine whether there is any excess portion of the biological material (i.e., excess sperm cells that are not required for the application use at milestone 8), and if that excess portion is going to be cryopreserved for subsequent use in further ART. The user interface 116″ receives input from the medical scientist identifying whether an excess portion of the biological material will undergo further cryopreservation, and the input device 112″ transmits this input to the coordination system 108, which processes the information before storing it to the blockchain in the same manner as described above in Example 1.

The coordination system 108 then receives confirmation from the gatekeeper that the data has been stored to the blockchain, and identifies that milestone 7 and subprocess 7.6 are complete. As the smart contract 124 dictates that the next milestone and subprocess to be achieved are milestone 8 and subprocess 8.1, the coordination system 108 writes to the blockchain to update the current status of the smart contract 124 to be milestone 8 subprocess 8.1 in the same manner described above in Example 1.

### Milestone 8

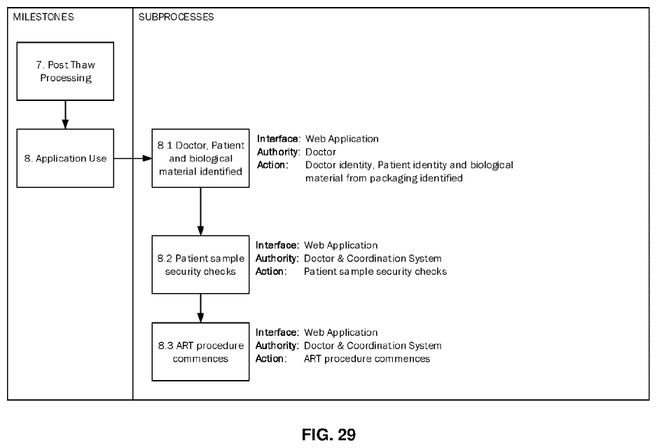

As shown in FIG. 29, an eighth milestone for this example is ‘Application Use’. At this milestone, at least a portion of the thawed biological material is used in an ART procedure, e.g., in vitro fertilization (IVF), Intracytoplasmic Sperm Injection (ICSI) or Intrauterine Insemination (IUI).

Based on the current status being subprocess 8.1 the coordination system 108 queries the data storage location 114 to retrieve executable instructions. In response, the coordination system 108 receives and executes executable instructions for subprocess 8.1, which in this example causes the processor 110 to do the following.

At subprocess 8.1, the coordination system 108 instructs the input device device 112 to inform the doctor (or other medical professional who is to perform or oversee the ART procedure) that they are required to provide status information in the form of doctor details (identifying the doctor), patient details (identifying the ART patient) and biological material details (identifying the biological material inside the packaging device 126). The input device 112 may provide the instructions to the doctor via the user interface 116. The details are input by the doctor using the user interface 116, and the input device 112 transmits the input status information to the coordination system 108. The coordination system 108 processes the received status information. The processing includes validating the status information to determine if the doctor details, patient details and biological material are valid for the smart contract 124. In this example, the validation may include querying the blockchain to check that the biological material matches the corresponding biological material stored on the blockchain. If the validation is successful, the coordination system 108 determines that the input doctor details, patient details and biological material are to be stored on the blockchain. If the validation is unsuccessful, the coordination system 108 instructs the input device to inform the doctor of the unsuccessful validation and request new input. The coordination system 108 may store a copy of the received status information in the data lake 128. Once processed, the coordination system 108 stores the status information to the blockchain (using the same steps described above in Example 1).

Upon receiving confirmation from the gatekeeper that the data has been stored to the blockchain, the coordination system 108 identifies that subprocess 8.1 is complete. As the smart contract 124 dictates that the next subprocess to be achieved is subprocess 8.2, the coordination system 108 writes to the blockchain to update the current status of the smart contract 124 to be subprocess 8.2 in the same manner described above in Example 1.

Based on the current status being subprocess 8.2, the coordination system 108 queries the data storage location 114 to retrieve executable instructions. In response, the coordination system 108 receives and executes executable instructions for subprocess 8.2, which in this example causes the processor 110 to do the following.

At subprocess 8.2, the coordination system 108 instructs the input device 112 to inform the doctor that the doctor and/or the patient is required to provide security check information for the biological material to be used in the ART procedure. The security check information may include a password and/or biometric information such as a retina or fingerprint scan to confirm the identity of the doctor and/or the patient. The input device 112 may provide the instructions to the doctor via the user interface 116. The details are input by the doctor and/or the patient (as required) using the user interface 116 (which may be configured to obtain the biometric information), and the input device 112 transmits the input status information to the coordination system 108. The coordination system 108 processes the received status information. The processing includes validating the status information to determine that the security check information is valid for the smart contract 124. If the validation is successful, the coordination system 108 determines that the success of the validation is to be stored on the blockchain. If the validation is unsuccessful, the coordination system 108 instructs the input device to inform the doctor of the unsuccessful validation and request new input, and determines that the failure of the validation is to be stored on the blockchain. Once processed, the coordination system 108 stores the relevant success or failure of the validation to the blockchain (using the same steps described above in Example 1).

Upon receiving confirmation from the gatekeeper that the data has been stored to the blockchain, the coordination system 108 identifies that subprocess 8.2 is complete. As the smart contract 124 dictates that the next subprocess to be achieved is subprocess 8.3, the coordination system 108 writes to the blockchain to update the current status of the smart contract 124 to be subprocess 8.3 in the same manner described above in Example 1.

Based on the current status being subprocess 8.3, the coordination system 108 queries the data storage location 114 to retrieve executable instructions. In response, the coordination system 108 receives and executes executable instructions for subprocess 8.3, which in this example causes the processor 110 to do the following.

At subprocess 8.3, the coordination system 108 instructs the input device 112 to inform the doctor to perform the ART procedure, e.g., through a message displayed on the user interface 116. The coordination system 108 subsequently identifies that milestone 8, subprocess 8.3 and hence the entire ART process defined by the smart contract 124 are complete.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method for controlling the handling of a biological material by a plurality of biological material handling apparatuses, the method implemented by a processor executing computer program instructions stored in memory and including the steps of comprising:
receiving, from one of:
a first apparatus of the plurality of apparatuses,
a device associated with a user interface for inputting data relating to biological material, and
a biological material packaging device,
status information for the biological material and identification information identifying a smart contract associated with the biological material;
querying a cryptographically secured distributed ledger using the identification information to retrieve the smart contract associated with the biological material, including a current status of the smart contract;
based on the retrieved smart contract and current status of the smart contract, querying a data storage location to retrieve executable instructions, wherein the data storage location is not a secure distributed ledger;
executing the executable instructions to cause the processor to at least:
process the status information, wherein the processing includes:
validating the status information, and
determining a part or parts of the status information to store on the distributed ledger; and
write to the distributed ledger to:
store the determined part or parts of the status information, and
update the status of the smart contract.

2. The method of claim 1, wherein executing the executable instructions further cause the processor to:
store the received status information to a data lake.

3. The method of claim 1, wherein the status information includes monitoring information for the biological material.

4. The method of claim 3, wherein the monitoring information includes at least one of temperature monitoring information, location monitoring information, volume monitoring information, and cell count information.

5. The method of claim 1, wherein the status information includes information about one or more previous handling steps taken in relation to the biological material.

6. The method of claim 1, wherein the biological material includes at least one of:
whole blood;
blood components;
stem cells;
modified cells;
gametes;
organs; and
tissues.

7. The method of claim 1, wherein executing the executable instructions further causes the processor to:
determine one or more commands or one or more computer executable instructions to be executed by a second apparatus of the plurality of apparatuses;
transmit the commands or computer executable instructions to the second apparatus.

8. The method of claim 7, wherein the commands or computer executable instructions are for controlling the second apparatus, and the second apparatus is one of the following:
a cryopreservation apparatus for cryopreserving the biological material;
a thawing apparatus for thawing the biological material;
a transport apparatus for transporting the biological material;
a storage apparatus for storing the biological material;
a cell counter apparatus for counting cells of the biological material.

9. The method of claim 1, wherein at least one apparatus of the plurality of apparatuses is configured to host at least one node of the distributed ledger.

10. The method of claim 9, further comprising determining that the at least one apparatus has sufficient processing and storage capacity to host the at least one node and execute the one or more commands or computer executable instructions, and in response allowing the at least one apparatus to host the at least one node.

11. The method of claim 1, further comprising:
receiving, from at least one of the first biological material handling apparatus and the biological material packaging device, historical status information and historical identification information;
comparing the historical status information and historical identification information with previously received status information and identification information to determine whether the previously received status information should be updated with the historical status information; and
in response to determining that the previously received status information should be updated with the historical status information, updating the previously received status information by storing an additional entry on the distributed ledger recording the historical status information.

12. The method of claim 1, further comprising:
receiving, from at least one of the first biological material handling apparatus and the biological material packaging device, historical status information and historical identification information;
determining from the historical identification information that the historical status information has not been previously received; and
storing the historical status information on the distributed ledger.

13. A method for controlling the handling of an extracted biological material from its initial extraction to its ultimate use, the handling involving multiple stages, the method including executing the method of claim 1 at a plurality of the multiple stages.

14. A method as claimed in claim 13, wherein the method of claim 1 is executed at each of the multiple stages.

15. A system for controlling the handling of a biological material comprising:
a plurality of biological material handling apparatuses;
a coordination system including:
a processor for executing computer program instructions; and
a memory storing computer program instructions for execution by the processor;
a cryptographically secured distributed ledger storing a smart contract associated with the biological material; and
a data storage location that is not a secure distributed ledger for storing retrievable computer-executable instructions;
wherein the computer program instructions stored in the memory include instructions which, when executed by the processor, cause the coordination system to:
receive status information for the biological material and identification information identifying the smart contract, from one of
a first apparatus of the plurality of apparatuses;
a device associated with a user interface for inputting data relating to the biological material; and
a biological material packaging device,
query the distributed ledger using the identification information to retrieve the smart contract and a current status of the smart contract; and
query the data storage location based on the smart contract and current status to retrieve computer-executable instructions; and
wherein the computer executable instructions stored in the data storage location include instructions which, when executed by the processor, cause the coordination system to:
process the status information by
validating the status information; and
determining a part or parts of the status information to store on the distributed ledger; and
write to the distributed ledger to store the determined part or parts of the status information and update the status of the smart contract.

16. A coordination system for controlling the handling of a biological material, the coordination system including:
at least one processor; and
a memory storing computer program instructions, which when executed by the at least one processor cause the coordination system to perform a method as claimed in claim 1.

17. A non-transitory, machine-readable storage medium having computer program instructions, which when executed by at least one processor of a coordination system cause the coordination system to perform a method as claimed in claim 1.
